(19) United States
(12) Patent Application Publication
(10) Pub. No.: US 2023/0105390 A1
(43) Pub. Date: Apr. 6, 2023

(54) SYSTEMS AND METHODS FOR DEPLOYMENT DETECTION OF ELECTROPORATION ABLATION CATHETERS

(71) Applicant: Boston Scientific Scimed Inc., Maple Grove, MN (US)

(72) Inventors: Sarah Renee Gutbrod, Medford, MA (US); Kurt Edmund Guggenberger, North Andover, MA (US); James Kauphusman, Champlin, MN (US)

(21) Appl. No.: 17/957,287

(22) Filed: Sep. 30, 2022

Related U.S. Application Data
(60) Provisional application No. 63/252,128, filed on Oct. 4, 2021.

(51) Int. Cl. A61B 34/20; A61B 18/14

(52) U.S. Cl. CPC A61B 34/20; A61B 18/1492; A61B 2018/1405; A61B 2018/00577

(57) ABSTRACT

At least some embodiments of the present disclosure are directed to systems and methods for estimating locations of electrodes and/or electrode assembly of an electroporation ablation catheter when the catheter is deployed. In some examples, the electrode position is estimated using electrical signals collected when a current is injected via tracking electrodes. In certain examples, the electrode positions are updated using one or more geometric models associated with the electroporation ablation catheter.

### CROSS REFERENCE TO RELATED APPLICATION

This application claims priority to U.S. Provisional Application No. 63/252,128, filed Oct. 4, 2021, which is incorporated herein by reference.

### TECHNICAL FIELD

The present disclosure relates to medical systems and methods for ablating tissue in a patient. More specifically, the present disclosure relates to medical systems and methods for ablation of tissue by electroporation.

### BACKGROUND

Ablation procedures are used to treat many different conditions in patients. Ablation can be used to treat cardiac arrhythmias, benign tumors, cancerous tumors, and to control bleeding during surgery. Usually, ablation is accomplished through thermal ablation techniques including radio-frequency (RF) ablation and cryoablation. In RF ablation, a probe is inserted into the patient and radio frequency waves are transmitted through the probe to the surrounding tissue. The radio frequency waves generate heat, which destroys surrounding tissue and cauterizes blood vessels. In cryoablation, a hollow needle or cryoprobe is inserted into the patient and cold, thermally conductive fluid is circulated through the probe to freeze and kill the surrounding tissue. RF ablation and cryoablation techniques indiscriminately kill tissue through cell necrosis, which may damage or kill otherwise healthy tissue, such as tissue in the esophagus, phrenic nerve cells, and tissue in the coronary arteries.

Another ablation technique uses electroporation. In electroporation, or electro-permeabilization, an electrical field is applied to cells in order to increase the permeability of the cell membrane. The electroporation can be reversible or irreversible, depending on the strength of the electric field. If the electroporation is reversible, the increased permeability of the cell membrane can be used to introduce chemicals, drugs, and/or deoxyribonucleic acid (DNA) into the cell, prior to the cell healing and recovering. If the electroporation is irreversible, the affected cells are killed through apoptosis.

Irreversible electroporation can be used as a nonthermal ablation technique. In irreversible electroporation, trains of short, high voltage pulses are used to generate electric fields that are strong enough to kill cells through apoptosis. In ablation of cardiac tissue, irreversible electroporation can be a safe and effective alternative to the indiscriminate killing of cells from thermal ablation techniques, such as RF ablation and cryoablation. Irreversible electroporation can be used to kill targeted tissue, such as myocardium tissue, by using an electric field strength and duration that kills the targeted tissue but does not permanently damage other cells or tissue, such as non-targeted myocardium tissue, red blood cells, vascular smooth muscle tissue, endothelium tissue, and nerve cells. Planning and/or facilitating electroporation ablation procedures can be difficult due to the lack of visualization or data indicating the location, the state, and/or shape of the catheter and electrode assembly before and during the ablation procedure.

### SUMMARY

In Example 1, a system for electroporation ablation comprises one or more tracking electrodes configured to deliver a tracking current, an ablation catheter including an electrode assembly, the electrode assembly including a plurality of splines and a plurality of electrodes, and one or more processors. At least one of the plurality of electrodes are disposed on the plurality of splines, and the ablation catheter is disposed proximate to target tissue; wherein the plurality of electrodes include a sensing electrode; wherein the sensing electrode is configured to measure electrical signals when the tracking current is delivered. The one or more processors may be configured to receive the measured electrical signals, estimate at least one electrode position corresponding at least one of the plurality of electrodes based on the measured electrical signals, and update, based on a geometric model of the ablation catheter, the at least one electrode position corresponding to at least one of the plurality of electrodes.

In Example 2, the system of Example 1, wherein the one or more processors are further configured to access a field map and estimate the at least one electrode position corresponding to at least one of the plurality of electrodes based on the measured electrical signals and the field map.

In Example 3, the system of Example 2, wherein the field map is generated by a mapping catheter.

In Example 4, the system of Example 2, wherein the ablation catheter further comprises a navigation sensor; wherein the one or more processors are configured to generate the field map based on sensing signals collected by the sensing electrode; wherein the sensing electrode has a known position relative to the navigation sensor.

In Example 5, the system of any of Examples 1-4, wherein the geometric model includes one or more constraints on one or more relative electrode positions of the plurality of electrodes.

In Example 6, the system of Example 5, wherein the geometric model includes a relative electrode position for two electrodes disposed on one spline of the plurality of splines.

In Example 7, the system of Example 5, wherein the geometric model includes a relative electrode position for two or more electrodes, and each electrode of the two or more electrodes may be disposed on a respective spline of the plurality of splines.

In Example 8, the system of Example 7, wherein the ablation catheter includes a longitudinal axis defined by a catheter shaft; wherein the electrode assembly extends from the catheter shaft; wherein the two or more electrodes form a plane generally perpendicular to the longitudinal axis.

In Example 9, the system of any of Examples 1-8, wherein the geometric model includes a first predetermined radius range of a first portion of a spline of the plurality of splines.

In Example 10, the system of Example 9, wherein the geometric model includes a second predetermined radius range of a second portion of the spline of the plurality of splines; wherein the second portion of the spline of the plurality of splines is different from the first portion of the spline of the plurality of splines; wherein the second predetermined radius range is different from the first predetermined radius range.

In Example 11, the system of any of Examples 1-10 further comprises a deployment sensor configured to collect data associated with a deployment state, wherein the one or more processors are configured to receive the collected data associated with the deployment state, and select the geometric model based on the collected data.

In Example 12, the system of any of Examples 1-11, wherein the one or more tracking electrodes includes a first tracking electrode configured to be disposed on a body surface of a patient.

In Example 13, the system of any of Examples 1-12, wherein the one or more tracking electrodes includes a second tracking electrode configured to be disposed in a cardiac chamber of a patient.

In Example 14, a method of electroporation ablations comprises deploying an ablation catheter proximate to target tissue, deploying one or more tracking electrodes to one or more target locations, injecting a current via the one or more tracking electrodes, measuring electrical signals via at least one of the plurality of electrodes, estimating an electrode position corresponding to one of the plurality of electrodes based on the measured electrical signals, and updating the electrode position based on a geometric model of the ablation catheter. The ablation catheter may include an electrode assembly, the electrode assembly including a plurality of splines and a plurality of electrodes, and at least one of the plurality of electrodes is disposed on the plurality of splines.

In Example 15, the method of example 14 further comprises accessing a field map, wherein the electrode position is estimated based on the measured electrical signals and the field map.

In Example 16, a system for electroporation ablation comprises one or more tracking electrodes configured to deliver a tracking current, an ablation catheter including an electrode assembly, the electrode assembly including a plurality of splines and a plurality of electrodes, and one or more processors. At least one of the plurality of electrodes are disposed on the plurality of splines, and the ablation catheter is disposed proximate to target tissue; wherein the plurality of electrodes include a sensing electrode; wherein the sensing electrode is configured to measure electrical signals when the tracking current is delivered. The one or more processors may be configured to receive the measured electrical signals, estimate at least one electrode position corresponding at least one of the plurality of electrodes based on the measured electrical signals, and update, based on a geometric model of the ablation catheter, the at least one electrode position corresponding to at least one of the plurality of electrodes.

In Example 17, the system of Example 16, wherein the one or more processors are further configured to access a field map and estimate the at least one electrode position corresponding to at least one of the plurality of electrodes based on the measured electrical signals and the field map.

In Example 18, the system of Example 17, wherein the field map is generated by a mapping catheter.

In Example 19, the system of Example 17, wherein the ablation catheter further comprises a navigation sensor; wherein the one or more processors are configured to generate the field map based on sensing signals collected by the sensing electrode; wherein the sensing electrode has a known position relative to the navigation sensor.

In Example 20, the system of Example 16, wherein the geometric model includes one or more constraints on one or more relative electrode positions of the plurality of electrodes.

In Example 21, the system of Example 20, wherein the geometric model includes a relative electrode position for two electrodes disposed on one spline of the plurality of splines.

In Example 22, the system of Example 20, wherein the geometric model includes a relative electrode position for two or more electrodes, and each electrode of the two or more electrodes may be disposed on a respective spline of the plurality of splines.

In Example 23, the system of Example 22, wherein the ablation catheter includes a longitudinal axis defined by a catheter shaft; wherein the electrode assembly extends from the catheter shaft; wherein the two or more electrodes form a plane generally perpendicular to the longitudinal axis.

In Example 24, the system of Example 16, wherein the geometric model includes a first predetermined radius range of a first portion of a spline of the plurality of splines.

In Example 25, the system of Example 24, wherein the geometric model includes a second predetermined radius range of a second portion of the spline of the plurality of splines; wherein the second portion of the spline of the plurality of splines is different from the first portion of the spline of the plurality of splines; wherein the second predetermined radius range is different from the first predetermined radius range.

In Example 26, the system of Example 16 further comprises a deployment sensor configured to collect data associated with a deployment state, wherein the one or more processors are configured to receive the collected data associated with the deployment state, and select the geometric model based on the collected data.

In Example 27, the system of Example 16, wherein the one or more tracking electrodes includes a first tracking electrode configured to be disposed on a body surface of a patient.

In Example 28, the system of Example 16, wherein the one or more tracking electrodes includes a second tracking electrode configured to be disposed in a cardiac chamber of a patient.

In Example 29, a method of electroporation ablations comprises deploying an ablation catheter proximate to target tissue, deploying one or more tracking electrodes to one or more target locations, injecting a current via the one or more tracking electrodes, measuring electrical signals via at least one of the plurality of electrodes, estimating an electrode position corresponding to one of the plurality of electrodes based on the measured electrical signals, and updating the electrode position based on a geometric model of the ablation catheter. The ablation catheter may include an electrode assembly, the electrode assembly including a plurality of splines and a plurality of electrodes, and at least one of the plurality of electrodes is disposed on the plurality of splines.

In Example 30, the method of example 29 further comprises accessing a field map, wherein the electrode position is estimated based on the measured electrical signals and the field map.

In Example 31, a system for electroporation ablation comprises one or more tracking electrodes configured to deliver a tracking current, an ablation catheter including an electrode assembly, the electrode assembly including a plurality of splines and a plurality of electrodes, and one or more processors. At least one of the plurality of electrodes are disposed on the plurality of splines, and the ablation catheter is disposed proximate to target tissue; wherein the plurality of electrodes include a sensing electrode; wherein the sensing electrode is configured to measure electrical signals when the tracking current is delivered. The electrode assembly has a plurality of deployment states, wherein the electrode assembly is in a first shape when the electrode assembly is at a first state of the plurality of deployment states; wherein the electrode assembly is in a second shape when the electrode assembly is at a second state of the plurality of deployment states; wherein the first state corresponds to a first geometric model, and the second state corresponds to a second geometric model. The one or more processors may be configured to receive the measured electrical signals, estimate at least one electrode position corresponding at least one of the plurality of electrodes based on the measured electrical signals, select a selected geometric model from the first geometric model and the second geometric model, and update, based on the selected geometric model of the ablation catheter, the at least one electrode position corresponding to at least one of the plurality of electrodes.

In Example 32, the system of Example 31, wherein the one or more processors are further configured to access a field map, and estimate the at least one electrode position corresponding to at least one of the plurality of electrodes based on the measured electrical signals and the field map.

In Example 33, the system of Example 31, wherein the geometric model includes one or more constraints on one or more relative electrode positions of the plurality of electrodes.

In Example 34, the system of Example 33, wherein the geometric model includes a relative electrode position for two electrodes of the plurality of electrodes disposed on one spline of the plurality of splines.

In Example 35, the system of Example 31 further comprises a deployment sensor configured to collect data associated with a deployment state. The one or more processors are configured to receive the collected data associated with the deployment state, and select the geometric model based on the collected data.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

FIG. 1 is a diagram illustrating an exemplary clinical setting for treating a patient, and for treating a heart of the patient, using an electrophysiology system, in accordance with embodiments of the subject matter of the disclosure.

FIGS. 2A-2B are schematic views illustrating an electroporation ablation catheter at various states that can be used for electroporation ablation, including ablation by irreversible electroporation, in accordance with embodiments of the subject matter of the disclosure.

FIGS. 3A-3C are schematic views illustrating an ablation catheter at various states that can be used for electroporation ablation, including ablation by irreversible electroporation, in accordance with embodiments of the subject matter of the disclosure.

FIGS. 4A-4D are schematic views illustrating an embodiment of ablation catheter that can be used for electroporation ablation, including ablation by irreversible electroporation, in accordance with embodiments of the subject matter of the disclosure.

FIGS. 5A-5D are schematic views illustrating a solid-core inductive sensor and an air-core inductive sensor, respectively, in accordance with embodiments of the subject matter of the disclosure.

FIG. 6 is a schematic view illustrating a catheter shaft.

FIGS. 7A-7B are schematic views illustrating an ablation catheter 700 including an electrode assembly and one or more tracking electrodes being deployed, in accordance with embodiments of the subject matter of the disclosure.

FIG. 8 is a flow chart diagram illustrating a process of facilitating ablation by irreversible electroporation, in accordance with embodiments of the subject matter of the disclosure.

FIGS. 9A-9E are flow diagrams and system diagrams illustrating processes of facilitating ablation by irreversible electroporation, in accordance with embodiments of the subject matter of the disclosure.

While the invention is amenable to various modifications and alternative forms, specific embodiments have been shown by way of example in the drawings and are described in detail below. The intention, however, is not to limit the invention to the particular embodiments described. On the contrary, the invention is intended to cover all modifications, equivalents, and alternatives falling within the scope of the invention as defined by the appended claims.

### DETAILED DESCRIPTION

The following detailed description is exemplary in nature and is not intended to limit the scope, applicability, or configuration of the invention in any way. Rather, the following description provides some practical illustrations for implementing exemplary embodiments of the present invention. Examples of constructions, materials, and/or dimensions are provided for selected elements. Those skilled in the art will recognize that many of the noted examples have a variety of suitable alternatives.

As the terms are used herein with respect to measurements (e.g., dimensions, characteristics, attributes, components, etc.), and ranges thereof, of tangible things (e.g., products, inventory, etc.) and/or intangible things (e.g., data, electronic representations of currency, accounts, information, portions of things (e.g., percentages, fractions), calculations, data models, dynamic system models, algorithms, parameters, etc.), “about” and “approximately” may be used, interchangeably, to refer to a measurement that includes the stated measurement and that also includes any measurements that are reasonably close to the stated measurement, but that may differ by a reasonably small amount such as will be understood, and readily ascertained, by individuals having ordinary skill in the relevant arts to be attributable to measurement error; differences in measurement and/or manufacturing equipment calibration; human error in reading and/or setting measurements; adjustments made to optimize performance and/or structural parameters in view of other measurements (e.g., measurements associated with other things); particular implementation scenarios; imprecise adjustment and/or manipulation of things, settings, and/or measurements by a person, a computing device, and/or a machine; system tolerances; control loops; machine-learning; foreseeable variations (e.g., statistically insignificant variations, chaotic variations, system and/or model instabilities, etc.); preferences; and/or the like.

Although illustrative methods may be represented by one or more drawings (e.g., flow diagrams, communication flows, etc.), the drawings should not be interpreted as implying any requirement of, or particular order among or between, various steps disclosed herein. However, some embodiments may require certain steps and/or certain orders between certain steps, as may be explicitly described herein and/or as may be understood from the nature of the steps themselves (e.g., the performance of some steps may depend on the outcome of a previous step). Additionally, a “set,” “subset,” or “group” of items (e.g., inputs, algorithms, data values, etc.) may include one or more items, and, similarly, a subset or subgroup of items may include one or more items. A “plurality” means more than one.

As used herein, the term “based on” is not meant to be restrictive, but rather indicates that a determination, identification, prediction, calculation, and/or the like, is performed by using, at least, the term following “based on” as an input. For example, predicting an outcome based on a particular piece of information may additionally, or alternatively, base the same determination on another piece of information.

Irreversible electroporation (IRE) uses high voltage, short (e.g., 100 microseconds or shorter) pulses to kill cells through apoptosis. IRE can be targeted to kill myocardium, sparing other adjacent tissues including the esophageal vascular smooth muscle and endothelium. IRE treatment can be delivered in multiple therapy sections. A therapy section (e.g., for a duration of 10 milliseconds) may include a plurality of electrical pulses (e.g., 20 pulses, 30 pulses, etc.) generated and delivered by an electroporation device, which is powered by an electroporation generator.

To determine the electrode positions and/or electrode assembly position of an electroporation ablation catheter in a conductive medium (e.g., an intracardiac space) using electric field localization technology (e.g., impedance tracking), in some embodiments, a system is configured to inject electric current to create an electric field and measure the resulting electric potential from the electrodes of an electroporation ablation catheter with an unknown 3D position. A tracking electrode with a surface exposed to the conductive media may be used to inject current. The surface of the tracking electrode may be disposed on the surface of the media (e.g., a patient's skin) or within the media (e.g., inside a patient's intravascular/intracardiac chamber). The system may collect electrical signals from one or more electrodes of the catheter when the current is injected via the tracking electrode.

Some mapping systems use the collected electrical signals in the context of a field map to determine positions of one or more electrodes and/or the electrode assembly and some mapping systems do this without the context of a field map. Electrodes of an electroporation ablation catheter may function as an ablation electrode for generating ablation electric field, a sensing electrode for measuring signals of an electric field, a mapping electrode for measuring electric signals to generate an electroanatomical map, a tracking electrode for injecting currents, and a combination thereof.

At least some embodiments of the present disclosure are directed to systems and methods for estimating locations, also referred to as positions, of electrodes and/or electrode assembly of an electroporation ablation catheter. At least some embodiments of the present disclosure are directed to systems and methods for estimating locations of electrodes and/or electrode assembly of an electroporation ablation catheter by tracking electrodes. In some examples, the electrodes are tracked using measured electrical signals when a current is injected via one or more tracking electrodes. In certain examples, the electrode positions are updated and/or refined using one or more geometric models corresponding to the electroporation ablation catheter.

As used herein, a geometric model refers to a mathematical model representing a shape, a shape associated with a range of changes, a predefined shape, an estimated shape, a predicted shape, a dynamic shape, an adjusted shape, a set of rules associated with one or more shapes, a set of rules associated with predetermined relative locations, a set of constraints associated with a shape, a set of constraints associated with predetermined relative locations, one or more geometric functions, and/or one or more functions relating relationships between components. In some embodiments, a geometric model is associated with a specific shape. As used herein, a shape refers to a two-dimensional shape or a three-dimensional shape of a specific size. In certain embodiments, a geometric model is associated with a plurality of shapes. In some embodiments, the systems and methods use the estimated positions associated with the electrodes and/or electrode assembly to facilitate ablation process. As used herein, “facilitating ablation” includes planning before an ablation procedure, providing localization information, and/or visualization guidance to assist ablation during the ablation procedure.

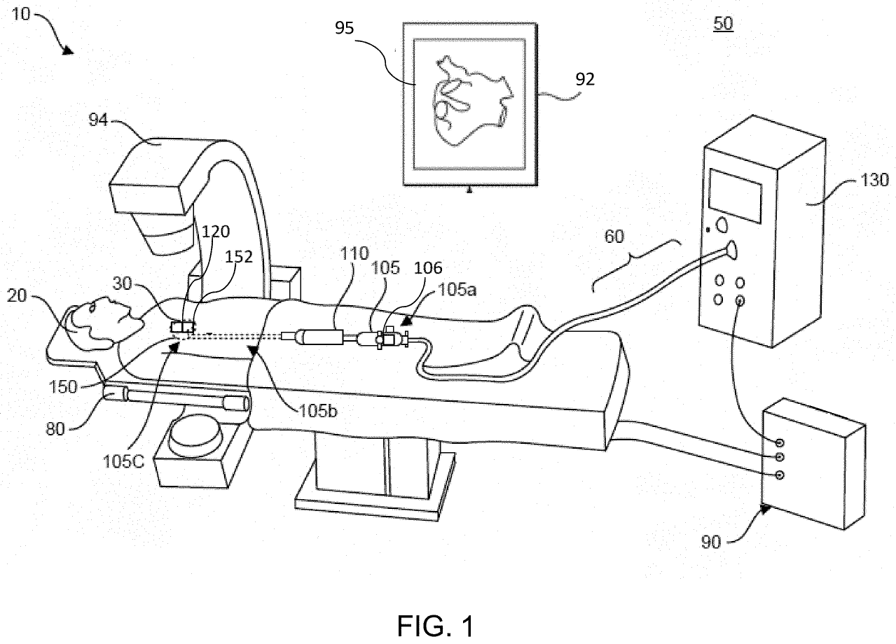

FIG. 1 is a diagram illustrating an exemplary clinical setting 10 for treating a patient 20, and for treating a heart 30 of the patient 20, using an electrophysiology system 50, in accordance with embodiments of the subject matter of the disclosure. The electrophysiology system 50 includes an electroporation device 60, a display 92, and an optional localization field generator 80. Also, the clinical setting 10 includes additional equipment such as imaging equipment 94 (represented by the C-arm) and various controlling elements configured to allow an operator to control various aspects of the electrophysiology system 50. As will be appreciated by the skilled artisan, the clinical setting 10 may have other components and arrangements of components that are not shown in FIG. 1.

The electroporation device 60 includes an electroporation catheter 105, an introducer sheath 110, a controller 90, and an electroporation generator 130. In embodiments, the electroporation device 60 is configured to deliver electric field energy to target tissue in the patient's heart 30 to create tissue apoptosis, rendering the tissue incapable of conducting electrical signals. In certain embodiments, the electroporation device 60 has a plurality of states, also referred to as operation states or deployment states, when used to ablate tissues. In some examples, the electroporation device 60 includes one or more tracking electrodes which can facilitate estimate and determining locations of electrodes of the electroporation catheter 105, locations of the electrode assembly 150 of the electroporation catheter 105, and/or the shape of the electrode assembly 150 of the electroporation catheter 105. In some embodiments, at least a part of the electrodes of the electroporation catheter 105 are ablation electrodes configured to generate electric fields for ablation during ablation procedures.

In some embodiments, the electroporation device 60 is configured to generate, based on models of electric fields, graphical representations of the electric fields that can be produced using the electroporation catheter 105 and to overlay, as presented on the display 92, the graphical representations of the electric fields on an anatomical map of the patient's heart to aid a user in planning and/or facilitating ablation (e.g., planning ablation before and facilitating during an ablation procedure by tracking locations of the electrode assembly 150) by irreversible electroporation using the electroporation catheter 105.

In embodiments, the electroporation device 60 is configured to generate the graphical representations of the electric fields based on characteristics of the electroporation catheter 105 and the position of the electroporation catheter 105 in the patient 20, such as in the heart 30 of the patient 20. In embodiments, the electroporation device 60 is configured to generate the graphical representations of the electric fields based on characteristics of the electroporation catheter 105 and the position of the electroporation catheter 105 in the patient 20, such as in the heart 30 of the patient 20, and the characteristics of the tissue surrounding the catheter 105, such as measured impedances of the tissue.

The controller 90 is configured to control functional aspects of the electroporation device 60. In embodiments, the controller 90 is configured to control the electroporation generator 130 to generate electrical pulses, for example, the magnitude of the electrical pulses, the timing and duration of electrics pulses. In embodiments, the electroporation generator 130 is operable as a pulse generator for generating and supplying pulse sequences to the electroporation catheter 105.

In embodiments, the introducer sheath 110 is operable to provide a delivery conduit through which the electroporation catheter 105 may be deployed to the specific target sites within the patient's heart 30. It will be appreciated, however, that the introducer sheath 110 is illustrated and described herein to provide context to the overall electrophysiology system 50.

As will be appreciated by the skilled artisan, the depiction of the electrophysiology system 50 shown in FIG. 1 is intended to provide a general overview of the various components of the system 50 and is not in any way intended to imply that the disclosure is limited to any set of components or arrangement of the components. For example, the skilled artisan will readily recognize that additional hardware components, e.g., breakout boxes, workstations, and the like, can and likely will be included in the electrophysiology system 50.

In the illustrated embodiment, the electroporation catheter 105 includes a handle 105a, a shaft 105b, and an electrode assembly 150. The handle 105a is configured to be operated by a user to position the electrode assembly 150 at the desired anatomical location. The shaft 105b has a distal end 105c and generally defines a longitudinal axis of the electroporation catheter 105. As shown, the electrode assembly 150 is located at or proximate the distal end 105c of the shaft 105b. In embodiments, the electrode assembly 150 is electrically coupled to the electroporation generator 130, to receive electrical pulse sequences or pulse trains, thereby selectively generating electrical fields for ablating the target tissue by irreversible electroporation.

In embodiments, as shown in FIG. 1, the electrode assembly 150 includes one or more electrodes 152. The electrodes 152 may include ablation electrodes, and optionally, mapping electrodes. In some configurations, the mapping electrodes are configured to be used to collect electrical signals to be used to generate, and display via the display 92, detailed three-dimensional geometric anatomical maps or representations of the cardiac chambers as well as electro-anatomical maps in which cardiac electrical activity of interest is superimposed on the geometric anatomical maps.

In certain embodiments, the electroporation catheter 105 is a catheter including an electrode assembly 150 that has a plurality of states. In embodiments, the electrode assembly 150 has a first shape when the catheter 105 is at a first state of the plurality of states, and a second shape when the catheter 105 is at a second state of the plurality of states. In some examples the electrode assembly 150 has more than two states (e.g., three states, five states, continuously varying states). In certain examples, the electrode assembly 150 has a respective contour (e.g., a contour having a shape different from another contour, a contour having a same shape but different size from another contour), also referred to as a respective shape. In some examples, the electrode assembly 150 includes one or more splines and one or more electrodes, where at least a part or all of the one or more electrodes are disposed on the one or more splines. In embodiments, at least a part of the one or more electrodes configured to generate ablation electric fields in a target tissue in response to a plurality of electrical pulse sequences.

In some embodiments, the electroporation catheter 105 includes a navigation sensor 120, or referred to as a set of navigation sensors, configured to collect sensor data associated with a location of the electrode assembly 150, location(s) of one or more components (e.g., shafts, tip, splines, electrodes, etc.) of the electrode assembly 150, and/or locations of one or more electrodes 152 of the electrode assembly 150. In certain embodiments, the sensor data collected by the navigation sensor 120 is measured when the localization field generator 80 is generating a magnetic field. In some embodiments, the navigation sensor 120 includes a first sensor disposed on one spline of the one or more splines. As used herein, the location of the electrode assembly 150 may refer to the location of one or more components of the electrode assembly 150. In some examples, the navigation sensor 120 collects electrical signals to facilitate determining a location of the navigation sensor 120, then further determining the location of the electrode assembly 150. In some embodiments, the electroporation catheter 105 includes a central shaft disposed in a cavity formed by the one or more splines, and the navigation sensor 120 includes a second sensor disposed in the central shaft. In certain embodiments, the electroporation catheter 105 further includes a catheter shaft, the electrode assembly 150 extending from the catheter shaft, and the navigation sensor 120 includes a third sensor (e.g., a catheter shaft sensor) disposed in the catheter shaft.

In embodiments, the navigation sensor 120 includes a 6-DOF (degree-of-freedom) sensor (e.g., a micro 6 DOF sensor). In some embodiments, the navigation sensor 120 includes an inductive sensor. In some embodiments, the navigation sensor 120 includes two 5-DOF sensors. In some examples, the navigation sensor 120 includes two 5-DOF sensors that is each disposed on a respective spline of the one or more splines of the electrode assembly 150. In certain examples, the navigation sensor 120 includes an inductive sensor integrated with a spline of the one or more splines. In some examples, the navigation sensor 120 includes an inductive sensor disposed at the central shaft. In certain examples, the navigation sensor 120 includes an magnetoresistive (MR) sensor disposed at a spline of the one or more splines, the central shaft, the distal end of the catheter shaft, and/or a distal cap of the electrode assembly 150.

In embodiments, the electroporation device 60 may include one or more tracking electrodes configured to deliver a current. The tracking electrode may include one or more electrodes disposed on the body surface of the patient 20 (e.g., on the back of the patient 20 or the chest of the patient 20), the intracardiac chamber of the patient 20, and/or one or more electrodes of the electroporation catheter 105.

In embodiments, the system 50 may include one or more sensing electrodes (e.g., one or more electrodes of the electroporation catheter 105) configured to measure electrical signals when the current is delivered by the tracking electrode. In embodiments, the controller 90 is configured to receive the measured electrical signals, estimate at least one electrode position corresponding to at least one of the one or more ablation electrodes based on the measured electrical signals, and update the at least one electrode position corresponding to at least one of the one or more ablation electrodes based on a geometric model of the ablation catheter. In certain embodiments, the controller 90 is configured to access a plurality of geometric models, where each geometric model is corresponding to a state of the electroporation catheter 105 and a predefined contour or shape of the electrode assembly 150 of the electroporation catheter 105.

In some embodiments, the electroporation device 60 includes one or more deployment sensors 106 configured to collect sensor data associated with a deployment state of the electroporation catheter 105. The one or more deployment sensors 106 may include a sensor disposed on the handle 105a (as illustrated) and/or a sensor disposed at the electrode assembly 150 (e.g., proximate to the cap of the electrode assembly 150) of the electroporation catheter 105. In some examples, the controller 90 is configured to determine a deployment state based on sensor data collected by the one or more deployment sensors 106. In certain examples, the controller 90 is configured to select a geometric model based on the determined deployment state. In some examples, the controller 90 is configured to select a geometric model based on the sensor data collected by the one or more deployment sensors 106 and the electrical signals measured by the one or more sensing electrodes.

In certain embodiments, the controller 90 is further configured to access a field map, and estimate the at least one electrode position corresponding at least one of the one or more ablation electrodes based on the measured electrical signals and the field map. In embodiments, the field map is generated by a separate mapping catheter. In embodiments, the field map is generated by mapping electrodes of the electroporation catheter 105.

In some embodiments, the one or more mapping electrodes on the electroporation catheter 105 can measure electrical signals and generate output signals that can be processed by the controller 90 to generate an electro-anatomical map, also referred to as an anatomical map. In some instances, electro-anatomical maps are generated before ablation for determining the electrical activity of the cardiac tissue within a chamber of interest. In some instances, electro-anatomical maps are generated after ablation in verifying the desired change in electrical activity of the ablated tissue and the chamber as a whole. The mapping electrodes may also be used to determine the position of the catheter 105 in three-dimensional space within the body. For example, when the operator moves the distal end of the catheter 105 within a cardiac chamber of interest, the boundaries of catheter movement can be used by the controller 90, which may include or couple to a mapping and navigation system, to form the anatomical map of the chamber. The chamber anatomical map may be used to facilitate navigation of the catheter 105 without the use of ionizing radiation such as with fluoroscopy, and for tagging locations of ablations as they are completed in order to guide spacing of ablations and aid the operator in fully ablating the anatomy of interest.

According to embodiments, various components (e.g., the controller 90) of the electrophysiological system 50 may be implemented on one or more computing devices. A computing device may include any type of computing device suitable for implementing embodiments of the disclosure. Examples of computing devices include specialized computing devices or general-purpose computing devices such as workstations, servers, laptops, portable devices, desktop, tablet computers, hand-held devices, general-purpose graphics processing units (GPGPUs), and the like, all of which are contemplated within the scope of FIG. 1 with reference to various components of the system 50.

In some embodiments, a computing device includes a bus that, directly and/or indirectly, couples the following devices: a processor, a memory, an input/output (I/O) port, an I/O component, and a power supply. Any number of additional components, different components, and/or combinations of components may also be included in the computing device. The bus represents what may be one or more busses (such as, for example, an address bus, data bus, or combination thereof). Similarly, in some embodiments, the computing device may include a number of processors, a number of memory components, a number of I/O ports, a number of I/O components, and/or a number of power supplies. Additionally, any number of these components, or combinations thereof, may be distributed and/or duplicated across a number of computing devices. In some embodiments, various components or parts of components (e.g., controller 90, electroporation catheter 105, etc.) can be integrated into a physical device.

In some embodiments, the system 50 includes one or more memories (not illustrated). The one or more memories includes computer-readable media in the form of volatile and/or nonvolatile memory, transitory and/or non-transitory storage media and may be removable, nonremovable, or a combination thereof. Media examples include Random Access Memory (RAM); Read Only Memory (ROM); Electronically Erasable Programmable Read Only Memory (EEPROM); flash memory; optical or holographic media; magnetic cassettes, magnetic tape, magnetic disk storage or other magnetic storage devices; data transmissions; and/or any other medium that can be used to store information and can be accessed by a computing device such as, for example, quantum state memory, and/or the like. In some embodiments, the one or more memories store computer-executable instructions for causing a processor (e.g., the controller 90) to implement aspects of embodiments of system components discussed herein and/or to perform aspects of embodiments of methods and procedures discussed herein.

Computer-executable instructions may include, for example, computer code, machine-useable instructions, and the like such as, for example, program components capable of being executed by one or more processors associated with a computing device. Program components may be programmed using any number of different programming environments, including various languages, development kits, frameworks, and/or the like. Some or all of the functionality contemplated herein may also, or alternatively, be implemented in hardware and/or firmware.

In some embodiments, the memory may include a data repository may be implemented using any one of the configurations described below. A data repository may include random access memories, flat files, XML files, and/or one or more database management systems (DBMS) executing on one or more database servers or a data center. A database management system may be a relational (RDBMS), hierarchical (HDBMS), multidimensional (MDBMS), object oriented (ODBMS or OODBMS) or object relational (ORDBMS) database management system, and the like. The data repository may be, for example, a single relational database. In some cases, the data repository may include a plurality of databases that can exchange and aggregate data by data integration process or software application. In an exemplary embodiment, at least part of the data repository may be hosted in a cloud data center. In some cases, a data repository may be hosted on a single computer, a server, a storage device, a cloud server, or the like. In some other cases, a data repository may be hosted on a series of networked computers, servers, or devices. In some cases, a data repository may be hosted on tiers of data storage devices including local, regional, and central.

Various components of the system 50 can communicate via or be coupled to via a communication interface, for example, a wired or wireless interface. The communication interface includes, but not limited to, any wired or wireless short-range and long-range communication interfaces. The wired interface can use cables, umbilicals, and the like. The short-range communication interfaces may be, for example, local area network (LAN), interfaces conforming known communications standard, such as Bluetooth® standard, IEEE 802 standards (e.g., IEEE 802.11), a ZigBee® or similar specification, such as those based on the IEEE 802.15.4 standard, or other public or proprietary wireless protocol. The long-range communication interfaces may be, for example, wide area network (WAN), cellular network interfaces, satellite communication interfaces, etc. The communication interface may be either within a private computer network, such as intranet, or on a public computer network, such as the internet. Various modifications and additions can be made to the exemplary embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

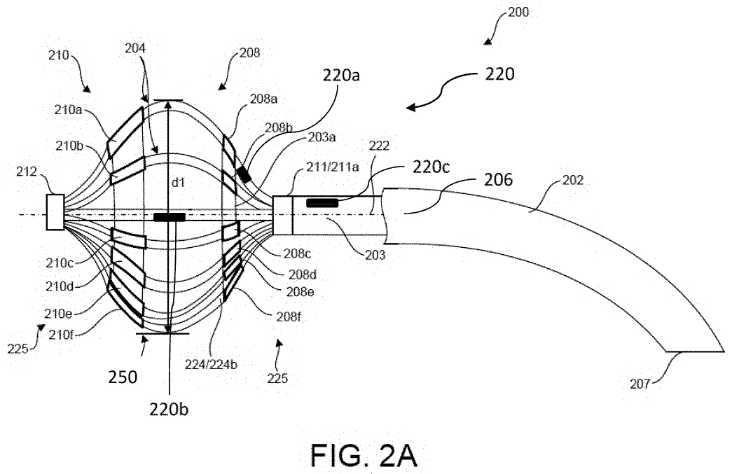

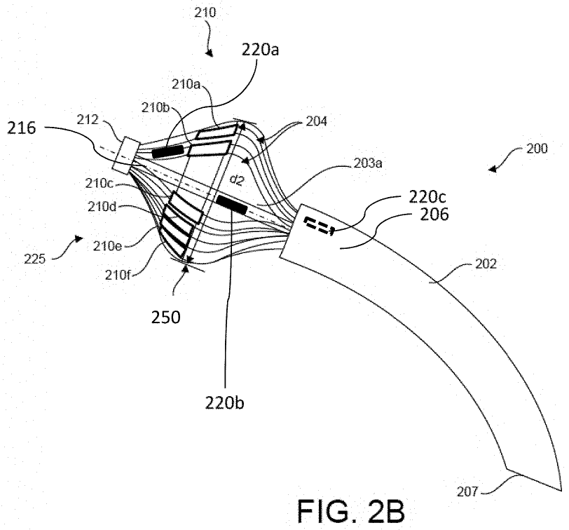

FIGS. 2A-2B are schematic views illustrating an electroporation ablation catheter 200 that can be used for electroporation ablation, including ablation by irreversible electroporation, in accordance with embodiments of the subject matter of the disclosure. FIG. 2A is a diagram illustrating the catheter 200 in a first state; FIG. 2B is a diagram illustrating the catheter 200 in a second state. The catheter 200 may have two or more states, where the states are either configurable or controllable by a user or automatically configurable by an electroporation system during treatment. The catheter 200 includes a catheter shaft 202 and a plurality of catheter splines 204 connected to the catheter shaft 202 at the distal end 206 of the catheter shaft 202. The catheter 200 may further include an inner shaft 203 disposed within the catheter shaft 202 and extending distally from a distal end 206 of the catheter shaft 202. As will be appreciated, the catheter shaft 202 is coupled, at its proximal end, to a handle assembly (not shown) configured to be manipulated by a user during an electroporation ablation procedure. As further shown, the catheter 200 includes an electrode assembly 250 at a distal end extending from the distal end 206 of the catheter shaft 202.

In embodiments, the electrode assembly 250 includes a plurality of energy-delivering electrodes (e.g., ablation electrodes) 225, where the electrode assembly 250 is configured to be selectively operable in a first state and a second state. In some cases, in the first state the electrode assembly 250 is configured to deliver ablative energy to form circumferential ablation lesion having a certain diameter.

In some embodiments, the electrode assembly 250 includes an inner shaft 203, where the inner shaft 203 is adapted to be extended from and retracted into the catheter shaft 202. In some cases, the electrode assembly 250 includes a plurality of splines 204 connected to the inner shaft 203 at a distal end 211 of the inner shaft 203. In some cases, the electrode assembly 250 further includes a central shaft 203a having a proximal end 211a (overlapped with the distal end 211 of the inner shaft 203) and a distal end 212. In some cases, the plurality of splines 204 are connected to the distal end 212 of the central shaft 203a. In embodiments, the electrodes 225 includes a plurality of first electrodes 208 and a plurality of second electrodes 210 disposed on the plurality of splines 204. In one example, the plurality of second electrodes 210 are disposed close to the distal end 212 of the central shaft 203a and the plurality of first electrodes 208 are disposed close to the proximal end 211a of the central shaft 203a.

In some cases, when operating in the first state, the inner shaft 203 and the central shaft 203a are extended from the catheter shaft 202, for example, as illustrated in FIG. 2A. In some cases, in the first state, both the plurality of first electrodes 208 and the plurality of second electrodes 210 are activated selectively energized to form relatively large diameter for circumferential ablation lesions, for example, used in a pulmonary vein isolation (PVI) procedure.

In some embodiments, when operating in the second state, the inner shaft 203 and the central shaft 203a are at least partially retracted into the catheter shaft 202 such that all or a part of the plurality of first electrodes 208 are retracted into the catheter shaft 202, for example, as illustrated in FIG. 2B. In some cases, in the second state, the plurality of first electrodes 208 are deactivated (e.g., by electrically disconnecting the first electrodes 208 from any pulse generator circuitry) and the plurality of second electrodes 210 are activated and used to create focal ablation lesions via electroporation.

The ablation catheter 200 has a longitudinal axis 222. As used herein, a longitudinal axis refers to a line passing through the centroid of the cross sections of an object. In embodiments, the plurality of splines 204 forms a cavity 224. The plurality of splines 204 forms a cavity 224a in the first state and forms a cavity 224b in the second state. In embodiments, the cavity 224a is larger than the cavity 224b in volume. In some embodiments, in the first state, the largest cross-sectional area generally perpendicular to the longitudinal axis 222 of the cavity 224a has a diameter d1. In some embodiments, in the second state, the largest cross-sectional area generally perpendicular to the longitudinal axis 222 of the cavity 224b has a diameter d2. In some cases, the diameter d1 is larger than the diameter d2.

In some examples, the diameter d1 is in the range of twenty (20) millimeters and thirty-five (35) millimeters. In certain examples, the diameter d1 is in the range of ten (10) millimeters and twenty-five (25) millimeters. In some examples, the diameter d2 is in the range of five (5) millimeters and sixteen (16) millimeters. In some examples, the diameter d2 is in the range of five (5) millimeters and sixteen (16) millimeters. In one example, the diameter d1 is greater than the diameter d2 by 30% to 100%. In one example, the diameter d1 is greater than the diameter d2 by at least 30%. In one example, the diameter d1 is greater than the diameter d2 by at least 20%. In one example, the diameter d1 is greater than the diameter d2 by at least 100% (i.e., at least two times of the diameter d2). In one example, the diameter dl is greater than the diameter d2 by at least 150% (i.e., at least two and a half times of the diameter d2).

In some cases, the first group of electrodes 208 disposed at or proximate the circumference of the plurality of splines 204 and the second group of electrodes 210 disposed proximate to the distal end 212 of the catheter 200. In some cases, the first group of electrodes 208 are referred to as proximal electrodes, and the second group of electrodes 210 are referred to as distal electrodes, where the distal electrodes 210 are disposed closer to the distal end 212 of the electroporation ablation catheter 200 than the proximal electrodes 208. In some implementations, the electrodes 225 can include a thin film of an electro-conductive or optical ink. The ink can be polymer-based. The ink may additionally comprise materials such as carbon and/or graphite in combination with conductive materials or a metal oxide coating, which could lower impedance on an electrode and increase signal to noise ratio. The electrode can include a biocompatible, low resistance metal such as silver, silver flake, gold, and platinum which are additionally radiopaque.

Each of the electrodes in the first group of electrodes 208 and each of the electrodes in the second group of electrodes 210 is configured to conduct electricity and to be operably connected to a controller (e.g., the controller 90 in FIG. 1) and an ablative energy generator (e.g., the electroporation generator 130 of FIG. 1). In embodiments, one or more of the electrodes in the first group of electrodes 208 and the second group of electrodes 210 includes flex circuits. In some cases, the plurality of first electrodes 208 are individually controllable. In some cases, the plurality of second electrodes are individually controllable. In some cases, all or a part of the plurality of first electrodes 208 are deactivated in the second state. In some cases, a part of the plurality of second electrodes 210 are deactivated in the second state.

Electrodes in the first group of electrodes 208 are spaced apart from electrodes in the second group of electrodes 210. The first group of electrodes 208 includes electrodes 208a-208f and the second group of electrodes 210 includes electrodes 210a-210f. Also, electrodes in the first group of electrodes 208, such as electrodes 208a-208f, are spaced apart from one another and electrodes in the second of electrodes 210, such as electrodes 210a-210f, are spaced apart from one another.

The spatial relationships and orientation of the electrodes in the first group of electrodes 208 and the spatial relationships and orientation of the electrodes in the second group of electrodes 210 in relation to other electrodes on the same catheter 200 is known or can be determined. In embodiments, the spatial relationships and orientation of the electrodes in the first group of electrodes 208 and the spatial relationships and orientation of the electrodes in the second group of electrodes 210 in relation to other electrodes on the same catheter 200 is constant, once the catheter is deployed. In embodiments, the spatial relationships and orientation of the electrodes in the first group of electrodes 208 and the spatial relationships and orientation of the electrodes in the second group of electrodes 210 in relation to other electrodes on the same catheter 200 is not constant. In some examples, the spatial relationships and orientation of the electrodes in the first group of electrodes 208 and the spatial relationships and orientation of the electrodes in the second group of electrodes 210 in relation to other electrodes on the same catheter 200 is predictable when the catheter is deployed.

As to electric fields, in embodiments, each of the electrodes in the first group of electrodes 208 and each of the electrodes in the second group of electrodes 210 can be selected to be an anode or a cathode, such that electric fields can be set up between any two or more of the electrodes in the first and second groups of electrodes 208 and 210. Also, in embodiments, each of the electrodes in the first group of electrodes 208 and each of the electrodes in the second group of electrodes 210 can be selected to be a biphasic pole, such that the electrodes switch or take turns between being an anode and a cathode. Also, in embodiments, groups of the electrodes in the first group of electrodes 208 and groups of the electrodes in the second group of electrodes 210 can be selected to be an anode or a cathode or a biphasic pole, such that electric fields can be set up between any two or more groups of the electrodes in the first and second groups of electrodes 208 and 210.

In embodiments, electrodes in the first group of electrodes 208 and the second group of electrodes 210 can be selected to be biphasic pole electrodes, such that during a pulse train including a biphasic pulse train, the selected electrodes switch or take turns between being an anode and a cathode, and the electrodes are not relegated to monophasic delivery where one is always an anode and another is always a cathode. In some cases, the electrodes in the first and second group of electrodes 208 and 210 can form electric fields with electrode(s) of another catheter. In such cases, the electrodes in the first and second group of electrodes 208 and 210 can be anodes of the fields, or cathodes of the fields.

Further, as described herein, the electrodes are selected to be one of an anode and a cathode, however, it is to be understood without stating it that throughout the present disclosure the electrodes can be selected to be biphasic poles, such that they switch or take turns between being anodes and cathodes. In some cases, one or more of the electrodes in the first group of electrodes 208 are selected to be cathodes and one or more of the electrodes in the second group of electrodes 210 are selected to be anodes. In embodiments, one or more of the electrodes in the first group of electrodes 208 can be selected as a cathode and another one or more of the electrodes in the first group of electrodes 208 can be selected as an anode. In addition, in embodiments, one or more of the electrodes in the second group of electrodes 210 can be selected as a cathode and another one or more of the electrodes in the second group of electrodes 210 can be selected as an anode.

In some instances, the first group of electrodes 208 is disposed proximal the maximum circumference (d1) of the catheter splines 204 and the second group of electrodes 210 disposed distal the maximum circumference of the catheter splines 204. In some embodiments, additional electrodes (e.g., mapping electrodes) may be added to each of the plurality of splines 204.

In embodiments, the ablation catheter 200 includes a navigation sensor 220 configured to collect sensor data associated with a location of the electrode assembly, the navigation sensor including a first sensor 220a disposed on one spline of the one or more splines 204. The location of the electrode assembly is associated with the location of the navigation sensor. In some embodiments, the ablation catheter 200 further includes a central shaft 203a disposed in a cavity formed by the one or more splines, and the navigation sensor 220 includes a second sensor 220b disposed in the central shaft 216. In some embodiments, the electroporation catheter 105 further includes a catheter shaft 202, the electrode assembly extending from the catheter shaft 202 at the distal end 206, and the navigation sensor 220 includes a catheter shaft sensor 220c disposed in the catheter shaft 202.

In some embodiments, the navigation sensor 220a and the second navigation sensor 220b are embedded in or integrated with the wall of a spline 204 and the central shaft 203a . In some embodiments, the navigation sensor 220 further includes a third navigation sensor 220c in addition to the first and second navigation sensor 220a, 220b. In some instances, the third navigation sensor 220c is disposed on the catheter shaft 202 (e.g., on the surface of the catheter shaft 202, within the catheter shaft 202). In certain instances, the third navigation sensor 220c, or referred to as the catheter shaft sensor, is dispose at the distal end 211 of the catheter shaft 202. In some instances, the third navigation sensor 220c may be disposed on one of the splines. In certain instance, the navigation sensors 220 includes sensors (e.g., inductive sensor, MR sensor, 5-DOF sensor, 6-DOF sensor) disposed on various components of the electroporation ablation catheter 200.

In embodiments, the navigation sensor 220 includes the navigation sensor 220a located on one of the splines and another navigation sensor (e.g., the third navigation sensor 200c) located on the catheter shaft 202. In embodiments, the navigation sensor 220a is a magnetoresistive sensor and the second navigation sensor 220b is an inductive sensor.

In some embodiments, the navigation sensor 220 includes a micro 6-DOF sensor. In some embodiments, the navigation sensor 220 includes one inductive sensor. In some embodiments, the navigation sensor includes one or more 5-DOF sensors and/or 6-DOF sensors.

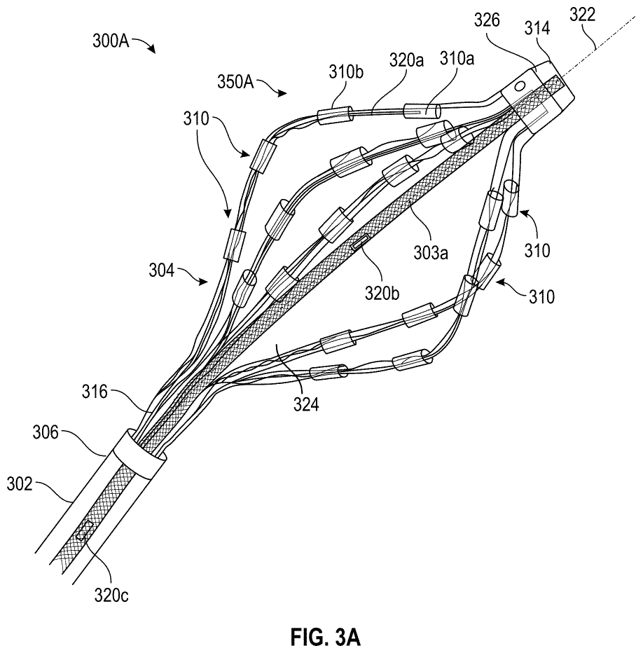

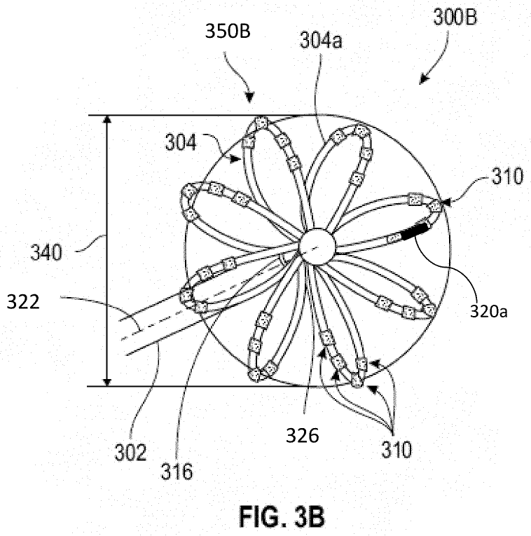

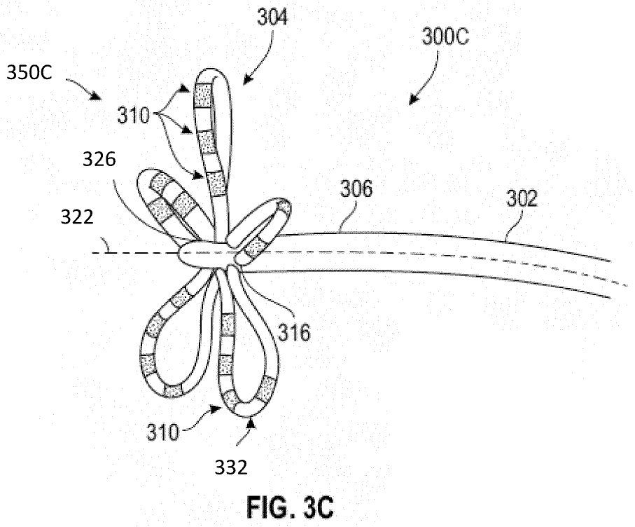

FIGS. 3A-3C are schematic views illustrating ablation catheter 300 at various states that can be used for electroporation ablation, including ablation by irreversible electroporation, in accordance with embodiments of the subject matter of the disclosure.

FIG. 3A shows a catheter 300A in a first state, or referred to as a first operation mode. In some embodiments, the catheter 300A includes an electrode assembly 350A. The electrode assembly 350A has a first shape, or referred to as a basket shape, in FIG. 3A. The catheter 300A includes a catheter shaft 302. The electrode assembly includes a plurality of splines 304 connected to the catheter shaft 302 at the distal end 306 of the catheter shaft 302. The catheter splines 304 includes a plurality of electrodes 310 disposed on the catheter splines 304. Each of the electrodes in the plurality of electrodes 310 is configured to conduct electricity and to be operably connected to the electroporation generator (e.g., the electroporation generator 130 in FIG. 1). In embodiments, one or more of the electrodes in the plurality of electrodes 310 includes metal.

The electrode assembly 350A has a proximal end 316 close to the distal end 306 of the catheter shaft 302 and a distal end 314 further away from the distal end 306 of the catheter shaft 302. As shown, the catheter shaft 302 defines a longitudinal axis 322, and the plurality of splines 304 are arranged in a curved shape between the distal end 314 and the proximal end 316. In embodiments, each spline 304 of the electrode assembly 350A in the first state is arranged as a curve with no turning point. In some examples, each spline 304 has a degree of curvature less than a predetermined degree. For example, each spline 304 has a degree of curvature less than 45°.

FIGS. 3B-3C shows a catheter 300B in a second state from an end view or referred to as a second operation mode; and FIG. 3C show a catheter 300C in a second state from a side view. In embodiments, each of the plurality of splines 304 includes one or more electrodes 310 disposed thereon. For example, as shown, spline 304a includes 4 electrodes. In some embodiments, each of the plurality of splines 304 may include more than 4 electrodes. In some embodiments, each of the plurality of splines 304 may include fewer than 4 electrodes. As may be understood by a skilled artisan, the number of electrodes on each spline may be adjusted, including the spacing between each electrode. The catheter shaft may further include a cap 326. In embodiments, the cap 326 is atraumatic to reduce trauma to tissue.

Each of the plurality of splines 304 as shown has similar size, shape, and spacing between adjacent electrodes 310 on a spline 304. In other embodiments, the size, shape, and spacing between adjacent electrodes 310 on a spline 304 may differ. In some embodiments, the thickness and length of each of the plurality of splines 304 may vary based on the number of electrodes and spacing between each electrode on the splines 304. The splines 304 may be made from similar or different materials, and may vary in thickness or length.

As shown, each of the plurality of splines 304 are arranged in a petal-like curve 332 in the second state, where the distal end 314 of the electrode assembly 350 is adjacent the proximal end 316 of the electrode assembly 350. Each of the plurality of splines 304 may pass through the distal end 306 of the catheter shaft 302 and be tethered to the catheter shaft 302 within a catheter shaft lumen. The distal end of each of the plurality of splines 304 may be tethered to the cap 326 of the catheter 300. In some embodiments, the one or more curve 332 are electrically isolated. As shown, the petal-like curve 332 includes a turning point.

In some embodiments, the catheter 300B includes an electrode assembly 350B arranged in a second shape, or referred to as a flower shape, as shown in FIG. 3B. In some embodiments, the catheter 300C includes an electrode assembly 350C arranged in the second shape as shown in FIG. 3C. As shown, each of the plurality of splines 304 may include a flexible curvature so as to rotate, or twist and bend and form the petal-shaped curve 332. The minimum radius of curvature of a spline in the petal-shaped configuration may be in the range of about 7 mm to about 25 mm. For example, the splines 304 may form an electrode assembly 350 at a distal portion of the catheter 300 and be configured to transform between a first shape where the set of splines are arranged generally parallel to the longitudinal axis of the catheter 300, and a second shape where the set of splines rotate around, or twist and bend, and generally bias away from the longitudinal axis of the catheter 300. In the first shape, each spline of the set of splines 304 may lie in one plane with the longitudinal axis 322. In the second shape, each spline of the set of splines 304 may bias away from the longitudinal axis 322 to form a petal-like curve 332 arranged generally perpendicular to the longitudinal axis 322. In this manner, the set of splines 304 twist and bend and bias away from the longitudinal axis 322 of the catheter 300, thus allowing the splines 304 to more easily conform to the geometry of an endocardial space, and particularly adjacent to the opening of a pulmonary ostium. The second shape may, for example, resemble the shape of a flower, from an end view as shown in FIG. 3B. In some embodiments, each spline in the set of splines in the second configuration may twist and bend to form a petal-like curve that, when viewed from front, displays an angle of curvature between the proximal and distal ends of the curve of proximate to 180 degrees.

The set of splines may further be configured to transform from a second shape to a third shape where the set of splines 304 may be apposed to (e.g., in contact with, or in apposition to) target tissue such as tissue surrounding a pulmonary vein ostium. The plurality of splines 304 may form a shape generally parallel to a longitudinal axis 322 of the catheter shaft 302 when undeployed, be wound (e.g., helically, twisted) about an axis (not shown) parallel to the longitudinal axis 322 when fully deployed, and form any intermediate shape (such as a cage or barrel) in-between the various the shapes. In some cases, when operating in first state, an inner shaft 303 including the central shaft 303a is extended from the catheter shaft 302, for example, as illustrated in FIG. 3A. In some cases, when operating in a second state, the inner shaft 303 is retracted into the catheter shaft 302, for example, as illustrated in FIGS. 3B-3C.

In embodiments, the ablation catheter 300 includes a navigation sensor 320 configured to collect sensor data associated with a location of the electrode assembly. In certain embodiments, the navigation sensor 320 is configured to collect sensor data associated with the location of the electrode assembly when a localization field generator (e.g., the localization field generator 80 in FIG. 1) is active. In some examples, the navigation sensor including a first navigation sensor 320a disposed on one spline of the one or more splines 304. The location of the electrode assembly is associated with the location of the navigation sensor. In some embodiments, the ablation catheter 300 further includes a central shaft 303a disposed in a cavity 324 formed by the one or more splines 304, and the navigation sensor 320 includes a second sensor 320b disposed in the central shaft 303a. In some embodiments, the ablation catheter further includes a catheter shaft 302, the electrode assembly extending from the catheter shaft 302 at the distal end 306, and the navigation sensor 320 includes a catheter shaft sensor 320c disposed in the catheter shaft 302.

In some embodiments, the navigation sensor 320a and the second navigation sensor 320b are embedded in the wall of the splines. In some embodiments, the navigation sensor 320 further includes a third navigation sensor 320c in addition to the first and second navigation sensor 320a, 320b. In some instances, the third navigation sensor 320c is disposed on the catheter shaft 302. In certain instances, the third navigation sensor 320c, or referred to as the catheter shaft sensor, is dispose at the distal end 306 of the catheter shaft 302. In some instances, the third navigation sensor 320c may be disposed on one of the splines. In certain instance, the navigation sensors 320 includes sensors (e.g., inductive sensor, MR sensor, 5-DOF sensor, 6-DOF sensor) disposed on various components of the electroporation ablation catheter 200.

In embodiments, the navigation sensor 320a is located on one of the splines, and another navigation sensor is located on the catheter shaft 302. In embodiments, the navigation sensor 320a is a magnetoresistive sensor and the navigation sensor 320b is an inductive sensor.

In some embodiments, the navigation sensor 320 includes a micro 6-DOF sensor. In some embodiments, the navigation sensor 320 includes one inductive sensor. In some embodiments, the navigation sensor includes one or more 5-DOF sensors and/or 6-DOF sensors.

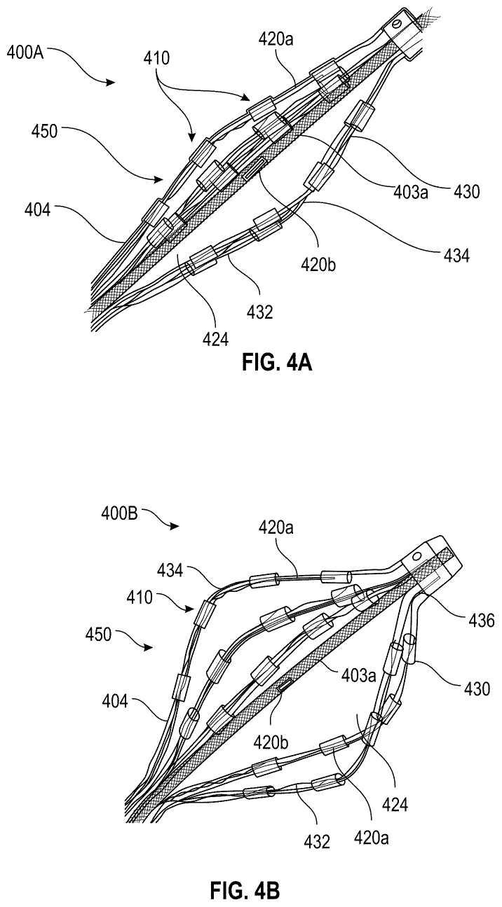

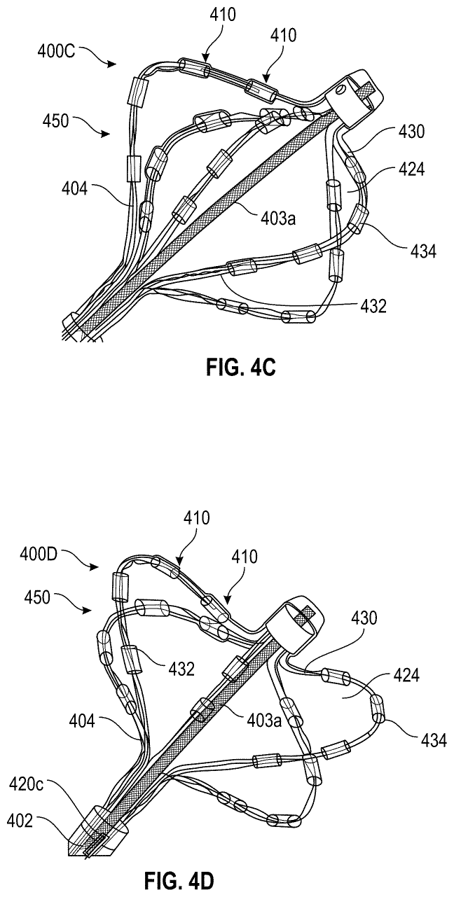

FIGS. 4A-4D are schematic views illustrating an embodiment of ablation catheter 400 that can be used for electroporation ablation, including ablation by irreversible electroporation, in accordance with embodiments of the subject matter of the disclosure.

FIG. 4A shows catheter 400A in a first state, or referred to as an undeployed state. FIG. 4B shows catheter 400B in a second state, or referred to as a deployed state 1. FIG. 4C shows catheter 400C in a third state, or referred to as a deployed state 2. FIG. 4D shows catheter 400D in a fourth state, or referred to as a deployed state 3.

As shown, the catheter 400 includes an electrode assembly 450 with one or more splines 404. In embodiments, the one or more splines 404 is a flat spline. As used herein, a flat spline has a thickness that is smaller than the width of the spline. In one example, the thickness of a flat spline is less than 75% of the width of the spline. As an example, the thickness of a flat spline is less than 60% of the width of the spline. In one example, the thickness of a flat spline is less than 50% of the width of the spline. As an example, the thickness of a flat spline is less than 25% of the width of the spline. In one example, the thickness of a flat spline is less than 10% of the width of the spline. In some examples, a catheter with flat splines has better flexibility, while the flat splines have challenges of housing certain components (e.g., sensor(s)). In embodiments, the electrode assembly includes one or more electrodes 410, at least a part of the one or more electrodes being disposed on the one or more splines, the one or more electrodes configured to generate electric fields in a target tissue in response to a plurality of electrical pulse sequences. In embodiments, the catheter 400 further includes a navigation sensor 420 configured to collect sensor data associated with a location of the electrode assembly, the navigation sensor 420 including a first sensor 420a disposed on one spline of the one or more splines.

The catheter 400 has a central shaft 403a disposed in a cavity 424 formed by the one or more splines 404. In some embodiments, the navigation sensor includes a second sensor 420b disposed in the central shaft 403a. In certain embodiments, the navigation sensor disposed in the central shaft 403a includes a micro 6-DOF sensor.

The catheter 400 also has a catheter shaft 402 where the electrode assembly 450 extends from. In embodiments, the navigation sensor includes a catheter shaft sensor 420c disposed in the catheter shaft 402. In some embodiments, the navigation sensor may include an inductive sensor. In certain embodiments, the navigation sensor may include two 5-DOF sensors. As understood by a person of skill in the art, there is no definitive correlation between the degrees of freedom (“DOF”) a certain sensor has and the type of sensor (e.g., inductive or magnetoresistive).

Each spline of the one or more splines 404 includes a first portion 430, and second portion 432, and a bending portion 434 connecting the first portion 430 and the second portion 432. As shown, when the catheter 400 is in various deployed states (e.g., deployed state 1, 2, and 3), the bending portion 434 is bent such that the first portion 430 and second portion 432 are closer or further apart in distance while remaining substantially straight compared to the bending portion 434. In some embodiments, the first portion 430 and/or the second portion 432 have a radius range that is smaller than the radius range of the bending portion 434.

In embodiments, a navigation sensor 420 may be disposed in the first portion 430 or second portion 432. Since the first and second portions 430, 432 remain substantially straight at one or more deployment states, the sensor will create less tension on the splines in each of their deployed states. During treatment through each of the deployed states, some potential problems created through too much tension include splines potentially breaking from tip adhesive point 436, or creating kinked wires within the spline. Decreasing tension created by disposing the navigation sensor in spline portions that remain substantially straight will help minimizing these problems from happening. Additionally, disposing the sensor in spline portions that remain substantially straight (e.g., the portions 430 and 432) will also in turn create less stress on the sensor , thereby reducing the chance of the sensor breaking and/or causing less changes to the sensor's electromagnetic properties, while the changes to the sensor's electromagnetic properties may lead to less accurate localization.

As mentioned above, a first sensor 420a may be disposed in one spline of the one or more splines. In embodiments, as will be discussed in more details below, a navigation sensor 420, or referred to as a set of navigation sensors, may be embedded in the wall of the one or more splines. The sensor embedded in the wall may be referred to as an air-core inductive sensor, as there is space in the middle of the coil. In embodiments, the navigation sensor may include a third sensor located on the catheter shaft 402. In other embodiments, the third sensor may be located on the one or more splines.

In embodiments, a first sensor may be located on one of the splines 404, and a second sensor may be located on the catheter shaft 402. The first sensor 420a may be a magnetoresistive sensor and the second sensor 420b may be an inductive sensor.

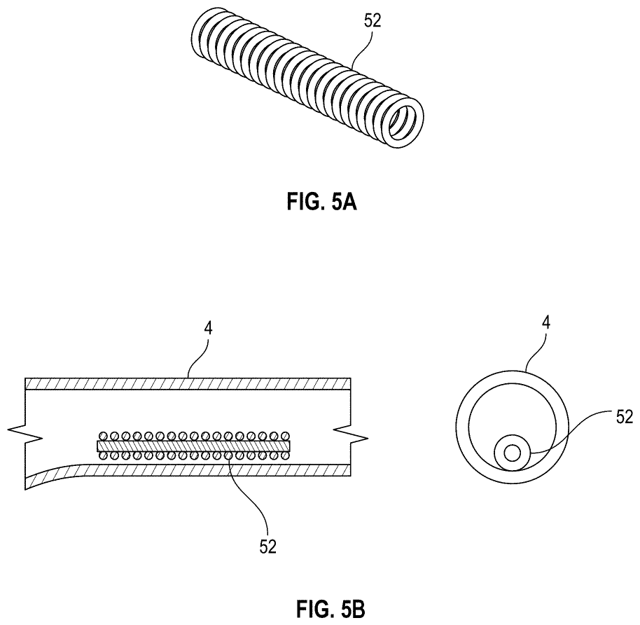

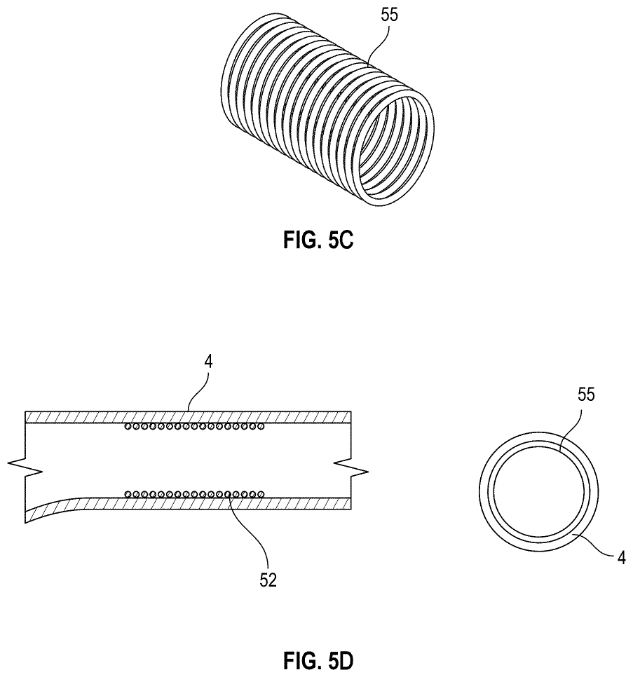

FIGS. 5A-5D are schematic views illustrating an inductive sensor and an air-core inductive sensor, respectively, in accordance with embodiments of the subject matter of the disclosure.

FIG. 5A illustrates an inductive sensor 52; and FIG. 5B shows two cross-sectional views of the inductive sensor 52 disposed in a supporting structure 4 (e.g., a spline, a central shaft, a catheter shaft). As shown in FIGS. 5A-5B, the inductive sensor 52 includes a plurality of turns of conductive wires . The coils are packed tightly so that the sensor 52 is smaller in size, and there is little to no space in the sensor formed. Due to the relative small size, the sensor 52 may fit into, and be disposed inside a supporting structure 4. In some examples, the sensor 52 is a solid-core inductive sensor.

FIG. 5C illustrates a sensor 55; and FIG. 5D shows two cross-sectional views of the sensor 55 disposed in or integrated with a supporting structure 4 (e.g., a spline, a central shaft, a catheter shaft). As shown in FIGS. 5C-D, the sensor 55 is an air-core inductive magnetic sensor with a plurality of turns of conductive wires. The coils forms a circle in the middle that has the approximately same radius as the opening in the middle of a supporting structure 4. In one example, the coils of conductive wire of the sensor 55 are embedded in the wall of the spline. As shown in the side view, the air-core inductive magnetic sensor 55 with the conductive wire is disposed circumferentially around the cavity of the supporting structure 4. In some instances, the sensor 55 may be disposed on a catheter shaft (e.g., the inner shaft 203 and the catheter shaft 202 in FIG. 2). This configuration advantageously maintains the patency of the spline opening to accommodate the passage of additional probes or devices. In some implementations, the sensor 55 allows one or more conductive wires to go through its air-core.

The internal payload space in devices can sometimes be partially obstructed by sensors such as the sensor 52 in FIG. 5A. An alternative sensor design (e.g. the air-core inductive sensor 55) of an air-core senor may reduce the obstruction on payload space of the device where the air-core sensor has an open center, thus enabling more payload to be integrated into the device.

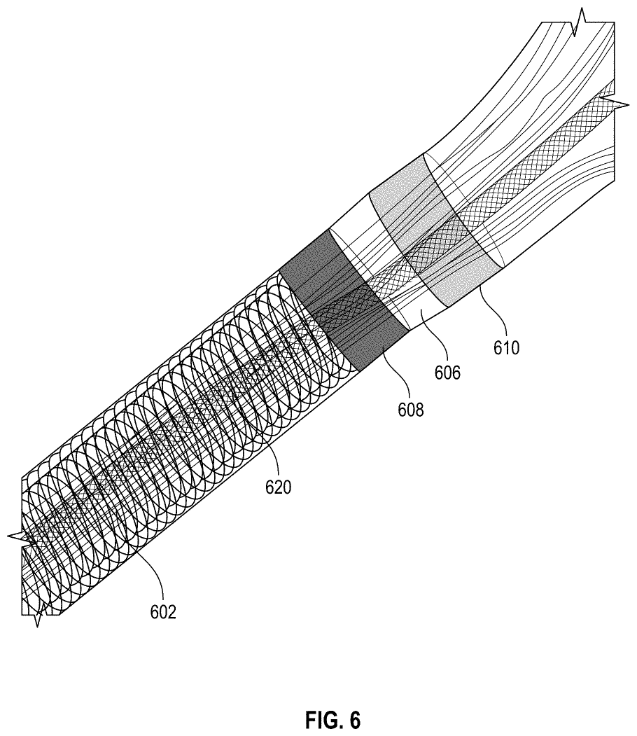

FIG. 6 is a schematic view illustrating a catheter shaft, in accordance with embodiments of the subject matter of the disclosure. As shown, the catheter shaft 602 includes a navigation sensor 620 located on the distal end 606 of the catheter shaft 602. The distal end 606 of the catheter shaft 602 is connected to the electrode assembly as shown in the previous figures. In embodiments, the navigation sensor 620 may be a 6 DOF sensor. In embodiments, the navigation sensor 620 may be a magnetoresistive sensor. In embodiments, the navigation sensor 620 may be an inductive sensor. In embodiments, the catheter shaft 602 may include a pull ring 608. In some instances, the catheter shaft 602 may include an electrode 610. The electrode 610 may be a tracking electrode to inject tracking current. In some embodiments, the electrode 610 may be a sensing electrode configured to collect electrical signals when the tracking current is injected during operation.

In embodiments, the navigation sensor 620 may be the only sensor located on the catheter shaft 602. In embodiments, the navigation sensor 620 may include a sensor in addition to and configured to work with other navigation sensors located on the electrode assembly (not shown).

In embodiments, the electrode 610 is a tracking electrode and the spatial relationship between the electrode 610 on the catheter is known with respect to the navigation sensor 620. The tracking electrode injects current to create a local electric field and corresponding signals measured by electrodes in the electrode assembly (e.g., the electrode assembly 350 in FIG. 3 or the electrode assembly 450 in FIG. 4) are used to detect the shape of the electrode assembly with respect to the tracking electrode 610 and navigation sensor 620, thereby resolving the global position and orientation of each electrode in the assembly. In one embodiment, the electrode 610 is a sensing electrode which has a known location with respect to the navigation sensor 620 is used to measure electrical signals used to generate a field map from the current injections of other tracking electrodes (e.g., tracking electrodes located on the skin of the patient). The generated field map is then used to track the locations of the electrodes in the electrode assembly.

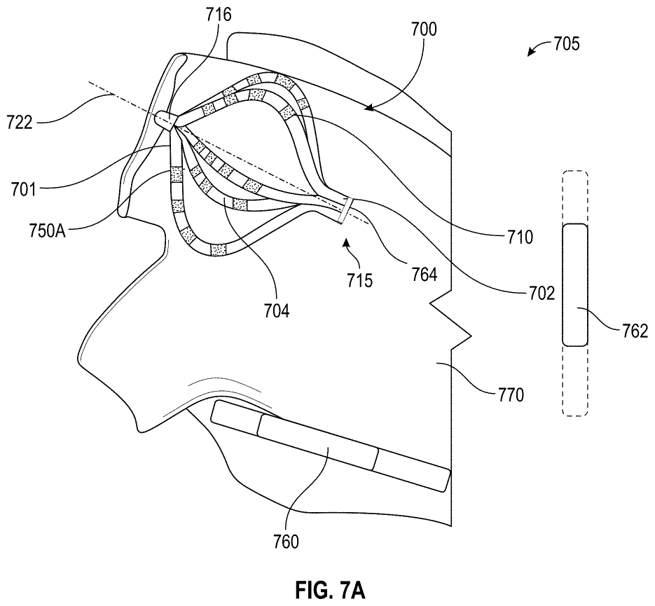

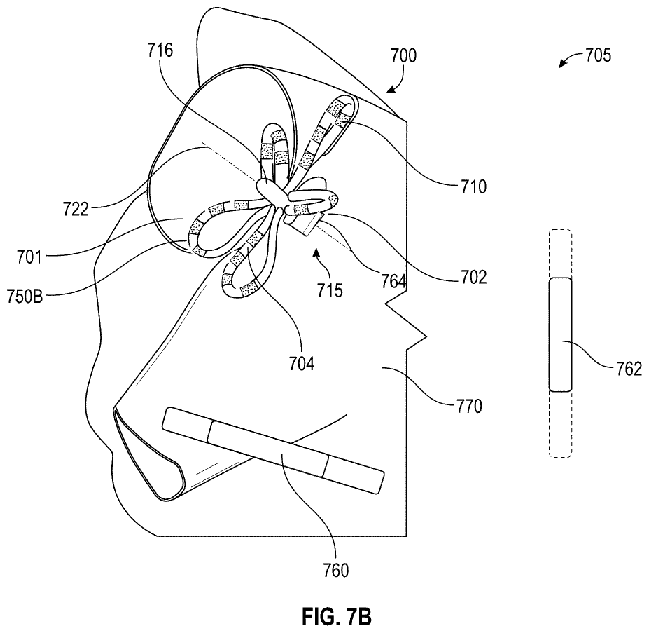

FIGS. 7A-7B are schematic views illustrating a system or an electroporation device 705 including an ablation catheter 700 with an electrode assembly and one or more tracking electrodes being deployed, in accordance with embodiments of the subject matter of the disclosure.

As shown, an electrode assembly 750 of an ablation catheter 700 is disposed proximate to a target tissue located in a patient's cardiac chamber 770. The electrode assembly 750 includes a plurality of splines 704 and a plurality of electrodes 710. At least one of the plurality of electrodes 710 is disposed on the plurality of splines 704. The electrodes assembly 750 may be in a first state as shown in FIG. 7A, or in a second state as shown in FIG. 7B. In embodiments, the catheter 700 includes a longitudinal axis 722 defined by a catheter shaft 702, and the electrode assembly 750 extends from the catheter shaft 702. In embodiments, the two or more electrodes 710 form a plane generally perpendicular to the longitudinal axis 722.

In embodiments, a system or electroporation device 705 for electroporation ablation may include an ablation catheter 700 including the electrode assembly 750. In embodiments, the system or electroporation device 705 for electroporation ablation may include one or more tracking electrodes 760, 762, 764 configured to deliver a current. As shown, the tracking electrode 760 may be disposed in a cardiac chamber 770 of a patient (e.g., an electrode on a catheter deployed in the cardiac chamber 770). In some embodiments, the tracking electrode 762 may be disposed on a body surface (not shown) of a patient (e.g., on the back or the chest of a patient). In some embodiments, the tracking electrode 764 may be disposed on the catheter shaft. In some embodiments, one of the electrodes 710 may be used as a tracking electrode for injecting current.

In embodiments, the system 705 for electroporation ablation includes one or more sensors (not shown) configured to measure electrical signals of at least one of the one or more electrodes 710 when the current is delivered. In embodiments, the system for electroporation ablation further includes one or more processors (not shown) configured to receive the measured electrical signals, estimate at least one electrode position corresponding at least one of the one or more electrodes 710 based on the measured electrical signals, and update the at least one electrode position corresponding at least one of the one or more electrodes 710 based on a geometric model of the ablation catheter 700.

In some embodiments, the system 705 is further configured to access a field map, and estimate the at least one electrode position corresponding at least one of the one or more electrodes 710 based on the measured electrical signals and the field map. In embodiments, the field map is generated by using a mapping catheter.

In embodiments, the ablation catheter 700 may include a navigation sensor or a set of navigation sensors (e.g., the navigation sensors shown in FIGS. 2-4), and the system 705 may be configured to generate the field map based on signals collected by the navigation sensor and a sensing electrode that has a fixed and known relationship with respect to the navigation sensor. In embodiment, the navigation sensor may be a 5-DOF sensor. In embodiment, the navigation sensor may be a 6-DOF sensor. In embodiment, the navigation sensor may be an inductive sensor. In embodiment, the sensing electrode is configured to measure the potential of current being injected.

In some embodiments, the system 705 uses one or more geometric models to determine and/or refine positions, also referred to as locations, of one or more electrodes in the electrode assembly 701 and/or the electrode assembly 701 after an initial estimate of the locations. In embodiments, the system 705 is configured to receive measured electrical signals when the tracking electrode (e.g., tracking electrode 760, tracking electrode 762) is injecting current, estimate at least one electrode position corresponding at least one of the one or more ablation electrodes based on the measured electrical signals, and update the at least one electrode position corresponding at least one of the one or more ablation electrodes or electrode assembly position based on a geometric model of the ablation catheter 700. In certain embodiments, the system 705 is configured to access a plurality of geometric models, where each geometric model is corresponding to a state of the electroporation catheter 700 and a predefined contour of the electrode assembly 701 of the electroporation catheter 700.

In certain embodiments, a geometric model includes rules applicable for catheters with a shape of splines (e.g., deformable splines). In some examples, a geometric model includes a rule of a range of radii, for example, which specify the curvature of the path between electrodes. In certain examples, a geometric model incudes an applicable rule represented by a function of the number of electrodes (e.g., path between electrodes 1 and 2 may have a different radius range from the path between electrodes 2 and 3).

In embodiments, the range of radii may be between adjacent electrodes. In some embodiments, the range of radii may be between the end points of each spline. In certain embodiments, a geometric model includes one or more rules representing tangency condition and/or volume of the cavity formed by the plurality of splines. In some embodiments, a geometric model includes a range of radii between the tip of the catheter 700 to an adjacent electrode (e.g., distal end 314 to first electrode 310a as shown in FIG. 3A), for example, the range of radii indicating a concave. In some embodiments, the range of radii may be between two adjacent electrodes (e.g., first electrode 310a to second electrode 310b as shown in FIG. 3A), and in a deployment state, the range of radii indicating a convex. In some embodiments, the range of radii may be between the first electrode (e.g., an electrode on a spline closest to the tip 716 of the catheter 700) and the last electrode (e.g., another electrode on the spline closest to the proximal end 715 of the catheter 700), where the shape would be substantially similar to a polynomial fit.

In some embodiments of the catheter 700 including flexible (e.g., deflectable) splines, the shape of each spline may not be identical to each other. The radius of a spline may change due to spline deformation by tissue contact. Therefore, the geometric model include rules (e.g., ranges of radii) for each spline respectively. In cases where spline deformation happens upon tissue contact, the system 705 may automatically and/or manually controlled by an operation to adjust the position of the electrode assembly 701 with the consideration of the deformation of one or more splines, resulting in deformation of the electrode assembly 701.

In embodiments, a geometric model may include one or more rules for electrodes on splines with the same order (e.g., electrode 1 on splines A, B, C; electrode 2 on splines A, B, C; electrode 3 on splines A, B, C; and electrode 4 on splines A, B, C). In some example, a geometric model may include a rule of electrodes on splines with the same order being on a same plane that is generally perpendicular to the longitudinal axis 722, or refer to as a same level of latitude. In certain embodiments, the system 705 is configured to apply the geometric model and adjust the electrode positions (e.g., snapping the electrodes from various splines to lie on the same level of latitude). In embodiments, the system 705 is configured to use the electrode positions to determine the shape of the electrode assembly and adjust electrode positions according to a template (e.g., a template for a deployment state).

In embodiments, a geometric model includes a rule (e.g., a constraint) including a predetermined relative position of the tip 716 of the catheter 700 with one more electrodes on a spline, for example, to avoid the tip 716 from permeating or damaging tissues during treatment. In embodiments where no electrode is located on the tip of the catheter, the tip may not be located by directly locating of an electrode, but may be located based on one or more rules (e.g., constraints) to provide updated and/or refined locations.

The geometric model may include one or more constraints on one or more relative electrode positions of the one or more ablation electrodes. In embodiments, the geometric model may include a relative electrode position for two ablation electrodes disposed on one spline of the one or more splines. In embodiments, the geometric model includes a relative electrode position for two or more ablation electrodes, each ablation electrode of the two or more ablation electrodes being disposed on a respective spline of the one or more splines.

In embodiments, the geometric model includes a first predetermined radius range of a first portion (e.g., the portion 430 in FIG. 4) of a spline of the one or more splines 704. In embodiments, the geometric model includes a second predetermined radius range of a second portion (e.g., the bending portion 434 in FIG. 4) of the spline of the one or more splines 704; the second portion of the spline of the one or more splines is different from the first portion of the spline of the one or more splines 704; and the second predetermined radius range is different from the first predetermined radius range.

In some embodiments, the system 705 for electroporation ablation includes a deployment sensor (e.g., the deployment sensor 106 in FIG. 1) configured to collect data associated with a deployment state. In embodiments, the system 705 is configured to receive the collected data associated with the deployment state, and update the geometric model or select a geometric model based on the collected data. In some instances, the system 705 is configured to update the geometric model by selecting a different geometric model. In certain instances, the system 705 is configured to update the geometric model by selecting a different geometric model corresponding to a deployment state. In embodiments, the deployment sensor may be located in a handle (e.g., the handle 105a shown in FIG. 1) or within an electrode assembly (e.g., the electrode assembly described in FIGS. 2-4). The handle 105a may include a slider that assists an operator in controlling the shape of the electrode assembly. For example, when the slider is pulled, the one or more splines on an electrode assembly is bent more and more to eventually turn into a petal-like shape (e.g., the electrode assemblies shown in FIGS. 2B-2C). When the slider is pushed, the one or more splines on an electrode assembly is bent less and less to return to a state where the one or more splines are substantially straight or relatively less bent. In some instances, the tip of the electrode assembly may twist around the longitudinal axis (e.g., axis 322 in FIG. 3). In certain embodiments, the collected data may be used to determine the degrees rotated at the tip of the electrode assembly, and based on the collected data, a deployment state may be determined to update the geometric model.

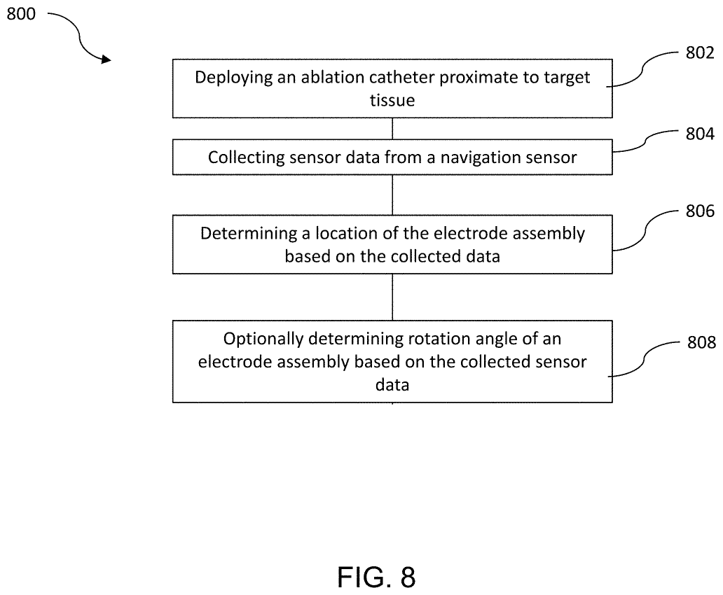

FIG. 8 is a flow chart diagram illustrating a process 800 of facilitating ablation by irreversible electroporation, in accordance with embodiments of the subject matter of the disclosure. The method is described in relation to the catheters discussed previously here, however, any suitable electroporation catheter can be used in the method. Aspects of embodiments of the method may be performed, for example, by an electrophysiology system or a controller (e.g., the system 50 in FIG. 1, the controller 90 in FIG. 1). One or more steps of method are optional and/or can be modified by one or more steps of other embodiments described herein. Additionally, one or more steps of other embodiments described herein may be added to the method.

At 802, the process 800 includes deploying an ablation catheter proximate to target tissue. The ablation catheter may include an electrode assembly and a navigation sensor. In embodiments, the electrode assembly includes a plurality of splines and a plurality of ablation electrodes, and at least one ablation electrode of the plurality of ablation electrodes is disposed on the plurality of splines. In embodiments, the navigation sensor is disposed on or integrated with at least on one spline of the plurality of splines.

At 804, the process 800 includes collecting sensor data from the navigation sensor. At 806, the process 800 includes determining a location of the electrode assembly based on the collected data. In embodiments, the electrode assembly state has a plurality of deployment states; the electrode assembly is in a first shape when the electrode assembly is at a first state of the plurality of deployment states, and the electrode assembly is in a second shape when the electrode assembly is at a second state of the plurality of deployment states.

At 808, the process 800 may optionally include determining a rotation angle of the electrode assembly based on the collected sensor data. In some embodiments, the navigation sensor may include two 5-DOF sensors. In some embodiments, the navigation sensor may include one 6-DOF sensor.

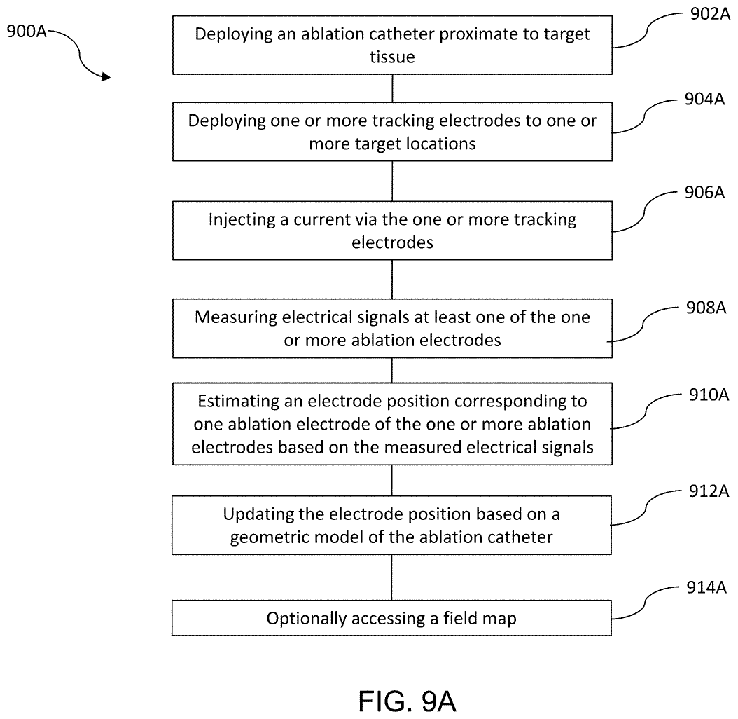

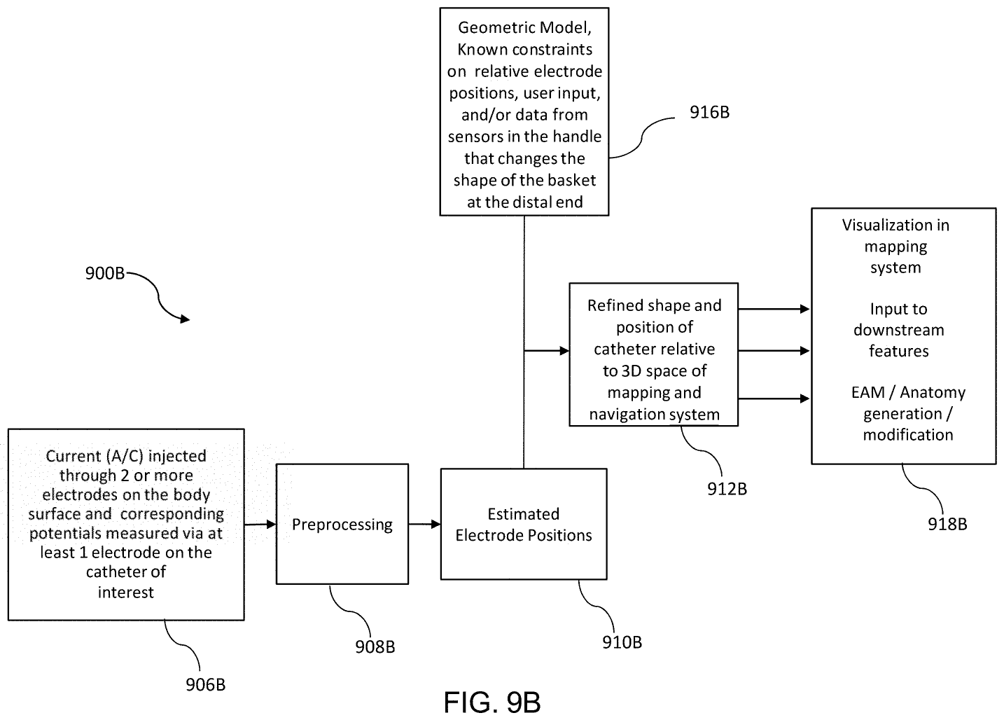

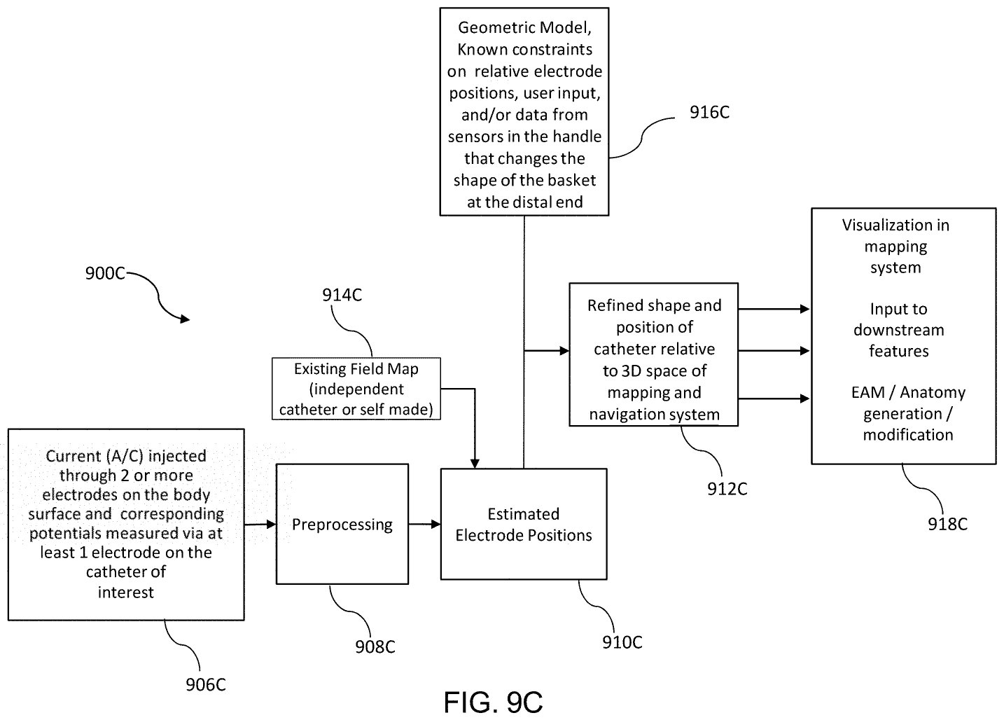

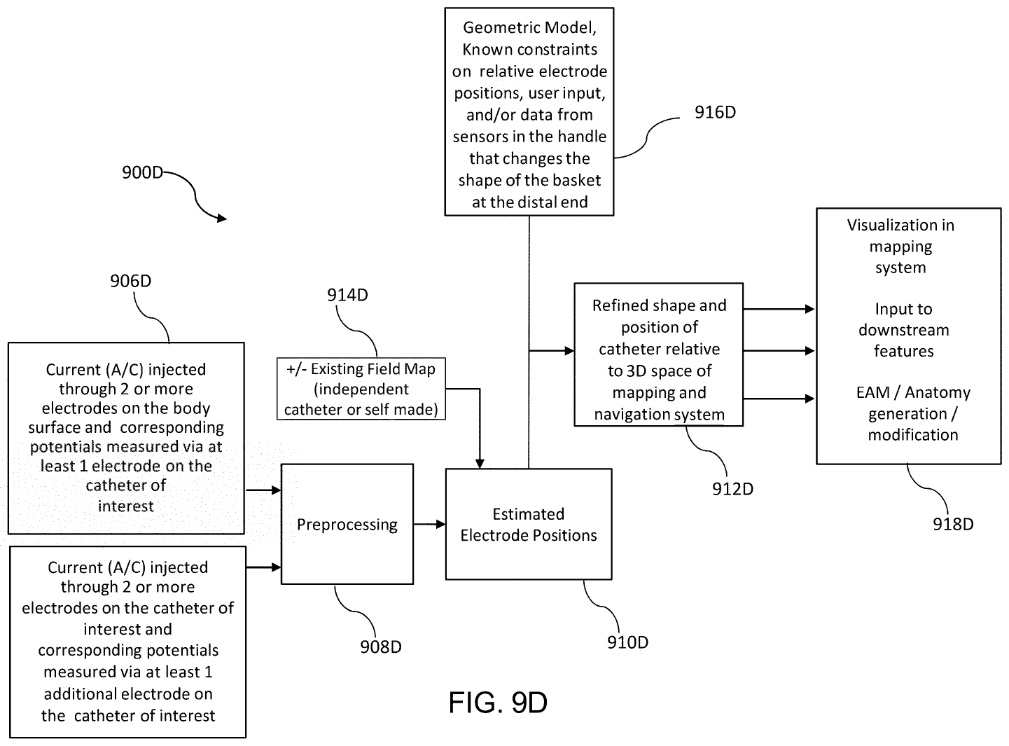

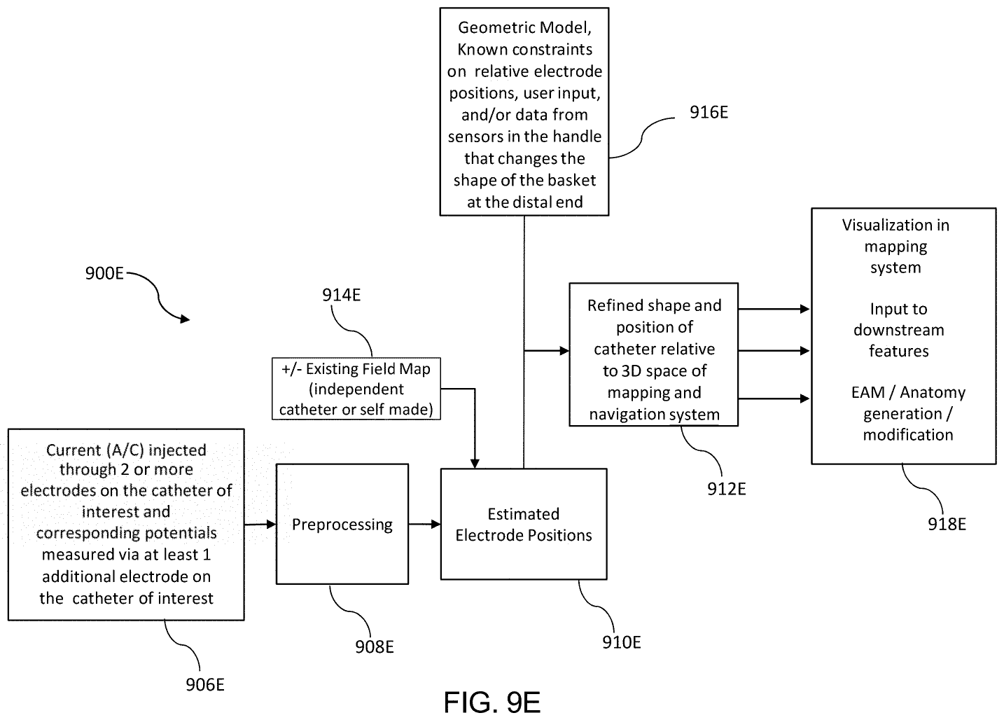

FIGS. 9A-9E are flow diagrams and system diagrams illustrating processes of facilitating ablation by irreversible electroporation, in accordance with embodiments of the subject matter of the disclosure. The method is described in relation to the catheters discussed previously here, however, any suitable electroporation catheter can be used in the method. Aspects of embodiments of the method may be performed, for example, by an electrophysiology system or a controller (e.g., the system 50 in FIG. 1, the controller 90 in FIG. 1). One or more steps of process are optional and/or can be modified by one or more steps of other embodiments described herein. Additionally, one or more steps of other embodiments described herein may be added to the example processes.

As illustrated in FIG. 9A, at 902A, the process 900A may include deploying an ablation catheter proximate to target tissue. In embodiments, the ablation catheter includes an electrode assembly, the electrode assembly including a plurality of splines and a plurality of ablation electrodes, and at least one of the plurality of ablation electrodes is disposed on the plurality of splines. At 904A, the process 900A may include deploying one or more tracking electrodes to one or more target locations.

At 906A, the process 900 may include injecting a current via the one or more tracking electrodes. At 908A, the process 900A may include measuring electrical signals via at least one of the one or more ablation.

At 910A, the process 900A may include estimating an electrode position corresponding to one ablation electrode of the one or more ablation electrodes based on the measured electrical signals. Various data sources may be used to estimate each individual electrode's position. For example, the data source may include potential measurements made from the catheter of interest relative to current injected by electrodes on the body surface.

In embodiments, the data source may include potential measurements made from the catheter of interest relative to current driven by separate electrodes on the catheter of interest. In some embodiments, the data source may include potential measurements made from a combination of injected current on both the body surface and local electrodes on the catheter of interest. In some embodiments, the data source may include potential measurements made from additional sensors on the ablation catheter (e.g., the navigation sensor in FIGS. 2-6).

Once individual electrode positions are estimated, tracking each electrode independently may compound the error of any tracking algorithm. In order to reduce this error, rules may be applied about the inter-electrode distance and the trajectory of the line drawn to connect electrodes together when displaying the catheter on a user interface. These rules adjust the individual 3D position of the electrodes within a mapping system. Rules may be applied for rigid linear catheters, flexible linear catheters, and/or existing commercial catheters (e.g., Orion). Rules may be more complex depending on the flexibility and shape of the catheter. At least some embodiments of this application include rules applicable for catheters with the shape of deformable splines.

At 912A, the process 900A may include updating the electrode position based on a geometric model of the ablation catheter.

In some embodiments, at 914A, the process 900A may optionally include accessing a field map, and the electrode position may be estimated based on the measured electrical signals and the field map. The field map may be an existing field map, for example, generated by a separate catheter, or generated by mapping electrodes on the ablation catheter.

FIGS. 9B-9E are system diagrams illustrating example processes of facilitating ablation by irreversible electroporation, in accordance with embodiments of the subject matter of the disclosure.

At 906B, the system 900B include injecting a current via two or more electrodes. There are various ways the current may be injected. For example, at 906B, 906C, the current may be injected through two or more electrodes on the body surface with corresponding electric potentials measured (“body surface dipole”) via at least one electrode on the catheter of interest. In embodiments, for example at 906D, the current may be injected through two or more electrodes on the body surface with corresponding electric potentials measured (“body surface and local dipole”) via at least one electrode on the catheter of interest, and through two or more electrodes on the catheter of interest with corresponding electric potentials measured via at least one additional electrode on the catheter of interest. In embodiments, for example at 906E, the current may be injected through two or more electrodes on the catheter of interest with corresponding potentials measured (“local dipole”) via at least one additional electrode on the catheter of interest.

At 908B-E, the systems 900B-E include preprocessing. In embodiments, preprocessing may include measuring electrical signals at one or more ablation electrodes.

At 910B-E, the systems 900B-E may include estimated electrode positions. The estimated electrode positions may include individual electrode positions, individual spline positions, and/or the position of the electrode assembly. Various data sources may be used to estimate each individual electrode's position. For example, the data source may include potential measurements made from the catheter of interest relative to current injected by electrodes on the body surface. This measurement may be made within the context of a field map (shown in FIG. 9C), optionally made within the context of a field map (shown in FIGS. 9D-E) or without a field map (shown in FIG. 9B). System 9B is an open-impedance tracking system since it does not rely on a field map.

In system 900C, also known as a closed-impedance tracking system, the measurement is required to be made within the context of a field map. In systems 900D-E, where the measurement is optionally made within the context of a field map, the systems are either an open or closed-impedance tracking system. As the field map is optional, 914D and 914E are marked with a “+/−” sign.

The field map may be made with an independent catheter or with an electrode on the shaft of the catheter of interest in a step wise approach (e.g., autologous field map creation).

In embodiments, as shown in FIGS. 9B-9E, the system for electroporation ablation includes a step 916B-E applying a geometric model. The geometric model may include one or more constraints on one or more relative electrode positions of the one or more ablation electrodes. In embodiments, the geometric model may include a relative electrode position for two ablation electrodes disposed on one spline of the one or more splines. In embodiments, the geometric model includes a relative electrode position for two or more ablation electrodes, each ablation electrode of the two or more ablation electrodes being disposed on a respective spline of the one or more splines.

In embodiments, the geometric model includes a first predetermined radius range of a first portion of a spline of the one or more splines. In embodiments, the geometric model includes a second predetermined radius range of a second portion of the spline of the one or more splines; the second portion of the spline of the one or more splines is different from the first portion of the spline of the one or more splines;

and the second predetermined radius range is different from the first predetermined radius range.

Applying the geometric model at 916B-E to the estimated electrode positions at 910B-E, the systems 900B-E may then determine a refined shape and position of the catheter relative to a 3D space of mapping and navigation system. The process of applying geometric models to estimated electrode positions may be repeated for a more accurate refined shape and position of the catheter.

In embodiments, the systems 900B-E may include one or more outputs 918B-E. The one or more outputs may include visualization in mapping system (e.g., on a display 92 in FIG. 1), input to downstream features, and/or EAM/anatomy generation/modification. In some embodiments, the outputs 918B-E can be used for real-time ablation planning and controlled. In certain embodiments, the outputs 918B-E can be used as inputs for a visualization system to provide real-time (e.g., within 1 second delay) information on the position, the shape, the orientation and other characteristics of the electrode assembly of the catheter.

Various modifications and additions can be made to the exemplary embodiments discussed without departing from the scope of the present disclosure. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

We claim:

1. A system for electroporation ablation, comprising:
at least one tracking electrode configured to deliver a tracking current;
an ablation catheter including an electrode assembly, the electrode assembly including a plurality of splines each including a plurality of ablation electrodes disposed thereon, the ablation catheter being configured such that the electrode assembly can be positioned proximate to target tissue, wherein the plurality of ablation electrodes are configured to measure electrical signals associated with the tracking current; and
one or more processors configured to:
receive the measured electrical signals;
estimate a position of each ablation electrode with respect to the at least one tracking electrode based on the measured electrical signals; and
determine a deployment state of the electrode assembly, based on a geometric model of the ablation catheter and the estimated positions of the ablations electrodes.

2. The system of claim 1, wherein the one or more processors are further configured to:
access a field map; and
estimate the electrode positions based on the measured electrical signals and the field map.

3. The system of claim 2, wherein the field map is generated by a mapping catheter.

4. The system of claim 2, wherein the ablation catheter further comprises a navigation sensor, wherein the one or more processors are configured to generate the field map based on sensing signals collected by the ablation electrode, wherein the ablation electrode has a known position relative to the navigation sensor.

5. The system of claim 1, wherein the geometric model includes one or more constraints on one or more relative positions of the plurality of ablation electrodes.

6. The system of claim 5, wherein the geometric model includes a relative position for two ablation electrodes disposed on one spline of the plurality of splines.

7. The system of claim 5, wherein the geometric model includes a relative electrode position for two or more ablation electrodes, each being disposed on a respective spline of the plurality of splines.

8. The system of claim 7, wherein the ablation catheter includes a longitudinal axis defined by a catheter shaft, wherein the electrode assembly extends from the catheter shaft, wherein the two or more ablation electrodes form a plane generally perpendicular to the longitudinal axis.

9. The system of claim 1, wherein the geometric model includes a first predetermined radius range of a first portion of a spline of the plurality of splines.

10. The system of claim 9, wherein the geometric model includes a second predetermined radius range of a second portion of the spline of the plurality of splines, wherein the second portion of the spline of the plurality of splines is different from the first portion of the spline of the plurality of splines, wherein the second predetermined radius range is different from the first predetermined radius range.

11. The system of claim 1, further comprising:
a deployment sensor configured to collect data associated with a deployment state;
wherein the one or more processors are configured to:
receive the collected data associated with the deployment state; and
select the geometric model based on the collected data.

12. The system of claim 1, wherein the at least one tracking electrodes includes a first tracking electrode configured to be disposed on a body surface of a patient.

13. The system of claim 1, wherein the at least one tracking electrodes includes a second tracking electrode configured to be disposed in a cardiac chamber of a patient.

14. A method of electroporation ablations, comprising:
deploying an ablation catheter proximate to target tissue, the ablation catheter including an electrode assembly, the electrode assembly including a plurality of splines each including a plurality of electrodes disposed thereon;
deploying one or more tracking electrodes to one or more target locations;
injecting a current via the one or more tracking electrodes;
measuring electrical signals via at least one of the plurality of electrodes associated with each of the plurality of splines;
estimating an electrode position corresponding the plurality of electrodes based on the measured electrical signals; and
updating the electrode position based on a geometric model of the ablation catheter.

15. The method of claim 14, further comprising:
accessing a field map;
wherein each electrode position is estimated based on the measured electrical signals and the field map.

16. A system for electroporation ablation, comprising:
one or more tracking electrodes configured to deliver a tracking current;
an ablation catheter including an electrode assembly, the electrode assembly including a plurality of splines each including a plurality of electrodes, the ablation catheter capable of being disposed proximate to a target tissue, wherein the plurality of electrodes includes a plurality of sensing electrodes, wherein the sensing electrodes are configured to measure electrical signals when the tracking current is delivered;
wherein the electrode assembly has a plurality of deployment states, wherein the electrode assembly is in a first shape when the electrode assembly is at a first state of the plurality of deployment states, wherein the electrode assembly is in a second shape when the electrode assembly is at a second state of the plurality of deployment states;
wherein the first state corresponds to a first geometric model, and the second state corresponds to a second geometric model; and
one or more processors configured to:
receive the measured electrical signals;
estimate each electrode position based on the measured electrical signals;
select a selected geometric model from the first geometric model and the second geometric model; and
determine a shape of the ablation catheter, based on the selected geometric model of the ablation catheter and the estimated electrode positions.

17. The system of claim 16, wherein the one or more processors are further configured to:
access a field map; and
estimate each electrode position based on the measured electrical signals and the field map.

18. The system of claim 16, wherein the geometric model includes one or more constraints on one or more relative electrode positions.

19. The system of claim 18, wherein the geometric model includes a relative electrode position for two electrodes of the plurality of the electrodes disposed on one spline of the plurality of splines.

20. The system of claim 16, further comprising:
a deployment sensor configured to collect data associated with a deployment state;
wherein the one or more processors are configured to:
receive the collected data associated with the deployment state; and
select the geometric model based on the collected data.
